(19) United States
(12) Patent Application Publication
(10) Pub. No.: US 2005/0075382 A1
(43) Pub. Date: Apr. 7, 2005

(54) N-Aryl-2-cyanooxazolidinones and their derivatives

(76) Inventors: Robert C. Gadwood, Portage, MI (US); Jason M. Ochoada, Greenwood, IN (US)

Correspondence Address: PHARMACIA & UPJOHN, 301 HENRIETTA ST, 0228-32-LAW, KALAMAZOO, MI, 49007, US

(21) Appl. No.: 10/917,937

(22) Filed: Aug. 13, 2004

Related U.S. Application Data
(60) Provisional application No. 60/497,181, filed on Aug. 22, 2003.

(51) Int. Cl. A61K 31/421; A61K 31/42; A61K 31/365

(52) U.S. Cl. 514/376; 514/378; 514/473; 548/229; 548/243; 549/321

(57) ABSTRACT

The present invention provides antibacterial agents having the formulae I, II, and III described herein.

### CROSS REFERENCE TO RELATED APPLICATIONS

This application claims the benefit of U.S. provisional application Ser. No. 60/497,181 filed Aug. 22, 2003, under 35 USC 119(e)(i), which are incorporated herein by reference in their entirety.

### FIELD OF THE INVENTION

The present invention relates to novel N-Aryl-2-cyanooxazolidinones, derivatives thereof, and their preparations. These compounds have potent antibacterial activity.

### BACKGROUND OF THE INVENTION

The oxazolidinone antibacterial agents are a novel synthetic class of antimicrobials with potent activity against a number of human and veterinary pathogens, including gram-positive aerobic bacteria such as multiply-resistant staphylococci and streptococci, anaerobic organisms such as bacteroides and clostridia species, and acid-fast organisms such as Mycobacterium tuberculosis and Mycobacterium avium.

### SUMMARY OF THE INVENTION

In one aspect, the invention provides compounds of formula I 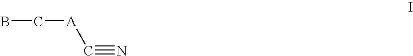
or a pharmaceutically acceptable salt thereof wherein:
A is a structure i, ii, or iii 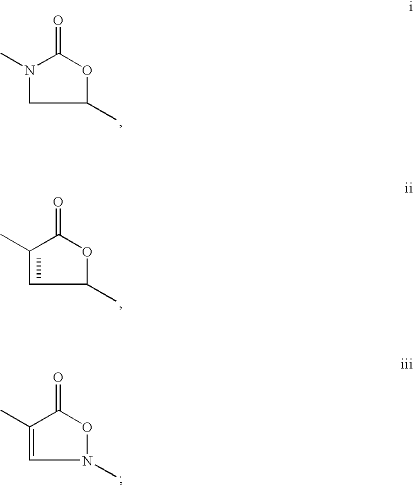
C is aryl or heteroaryl, wherein each of the aryl and heteroaryl are optionally substituted with 1-3 of R₂;
B is selected from cycloalkyl, substituted cycloalkyl, cycloalkenyl, substituted cycloalkenyl, aryl, substituted aryl, het and substituted het, or B and one R₂, if present, together, with the phenyl carbon atoms to which B and the one R₂ are bonded, form a het, the het optionally being a substituted het,
Each R₂ is independently selected from H, alkyl, amino, NO₂, —CN, halo, and substituted alkyl; and
Each R₃ is independently selected from H or C₁-C₆ alkyl.

Embodiments of this aspect of the invention may include one or more of the following features. Each R₂ is independently selected from H, F, Cl, Br, CN, NH₂, NO₂, CF₃, and CH₃. The structure of A is 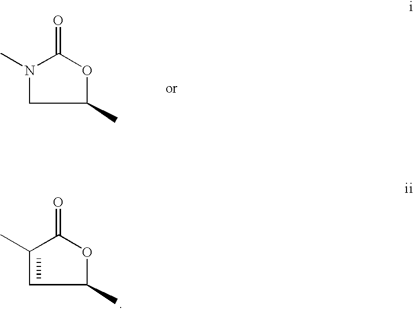
B is het or substituted het such as morpholinyl, piperazinyl, pyridyl, thiomorpholinyl, 3,6-dihydro-2H-thiopyranyl, tetrahydro-2H-thiopyranyl, 3,6-dihydro-2H-pyranyl, tetrahydro-2H-pyranyl, azetidinyl, 5,6-dihydro-4H-[1,3,4]thiadiazinyl, 2,5-dihydro-1H-pyrrolyl, 3,4-dihydro-1(2H)-pyridinyl, tetrahydropyridyl, 5,7-dihydro-6H-pyrrolo[3,4-b]pyridinyl, 2,3-dihydro-4H-1,4-thiazinyl, each of the morpholinyl, piperazinyl, pyridyl, thiomorpholinyl, 3,6-dihydro-2H-thiopyranyl, tetrahydro-2H-thiopyranyl, 3,6-dihydro-2H-pyranyl, tetrahydro-2H-pyranyl, azetidinyl, 5,6-dihydro-4H-[1,3,4]thiadiazinyl, 2,5-dihydro-1H-pyrrolyl, 3,4-dihydro-1(2H)-pyridinyl, tetrahydropyridyl, 5,7-dihydro-6H-pyrrolo[3,4-b]pyridinyl, 2,3-dihydro-4H-1,4-thiazinyl being optionally substituted with 1-4 groups selected from ═O, alkyl, substituted alkyl, amino, substituted amino, —OH, ═NOH, ═NC₁₋₄ alkyl, and halo.
B is selected from 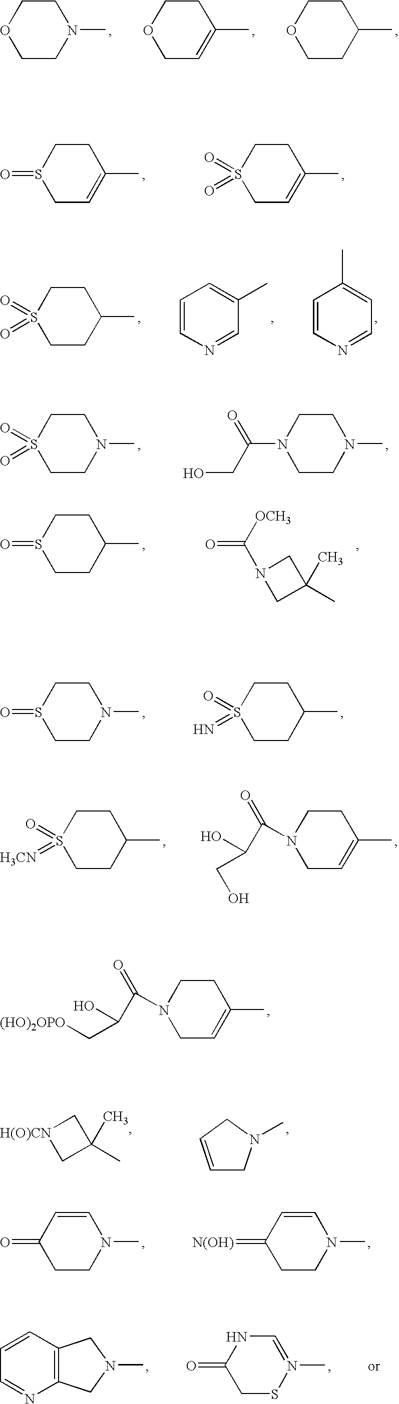 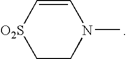
One R₂ is hydrogen and the other R₂ is F. Both R₂ substituents are F. One R₂ and B together form a het. R₂ and B form —S—C(O)—N(Q₅₀)-, —O—C(O)—N(Q₅₀)-, —N(Q₅₀)-HCQ₅₀-CH₂—, —NQ₅₀-C(O)—CH₂—O—, —NQ₅₀-C(O)—CF₂—O—, —NQ₅₀-C(O)—CH₂—S—, —NQ₅₀-C(O)—CF₂—S—, —NQ₅₀-C(S)—CH₂—S—, —NQ₅₀-C(O)—CH₂—CH₂—, —CH₂—CH₂—NQ₅₀-CH₂—CH₂—, or —CH₂—NQ₅₀-CH₂—CH₂—CH₂—, where Q₅₀ is H or C₁₋₄ alkyl optionally substituted with 1-3 of ═O, or —OH. Q₅₀ is methyl, isopropyl, ethyl, formyl, acetyl, or —C(O)—CH₂OH.

In another aspect, the invention provides compounds of formula II 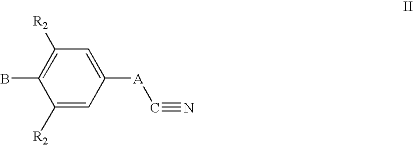
or a pharmaceutically acceptable salt thereof wherein:
A is a structure i, ii, or iii 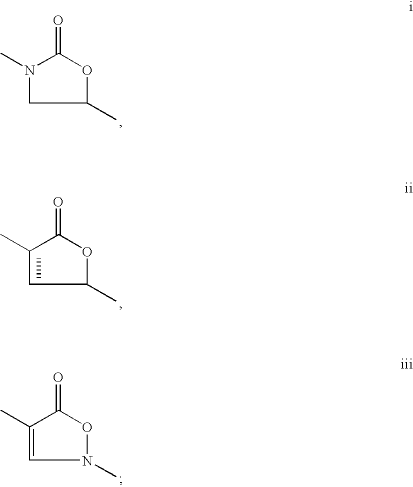
B is selected from cycloalkyl, substituted cycloalkyl, cycloalkenyl, substituted cycloalkenyl, aryl, substituted aryl, het, and substituted het, or B and one R₂ together, with the phenyl carbon atoms to which B and the one R₂ are bonded, form a het, the het optionally being a substituted het,
Each R₂ is independently selected from H, alkyl, amino, NO₂, —CN, halo, and substituted alkyl;
Each R₃ is independently selected from H or C₁-C₆ alkyl.

In another aspect the invention features a compound of formula III 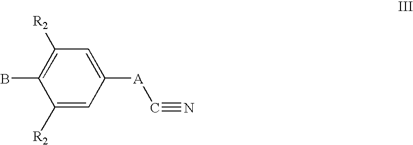
or a pharmaceutically acceptable salt thereof wherein:
A is a structure i, ii, or iii
A is a structure i, ii, or iii 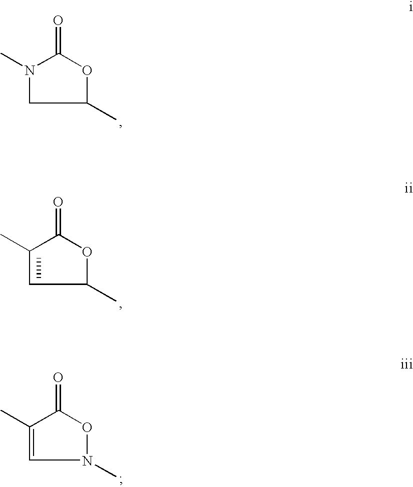
B is 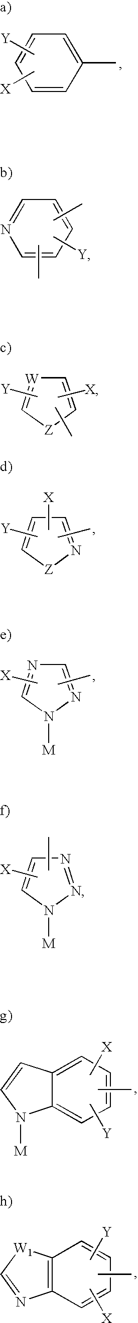 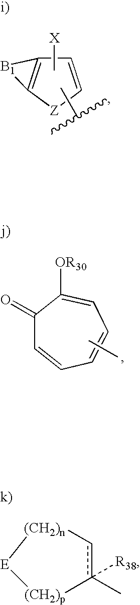
   l) a diazinyl group optionally substituted with X and Y,
   m) a triazinyl group optionally substituted with X and Y,
   n) a quinolinyl group optionally substituted with X and Y,
   o) a quinoxalinyl group optionally substituted with X and Y,
   p) a naphthyridinyl group optionally substituted with X and Y, 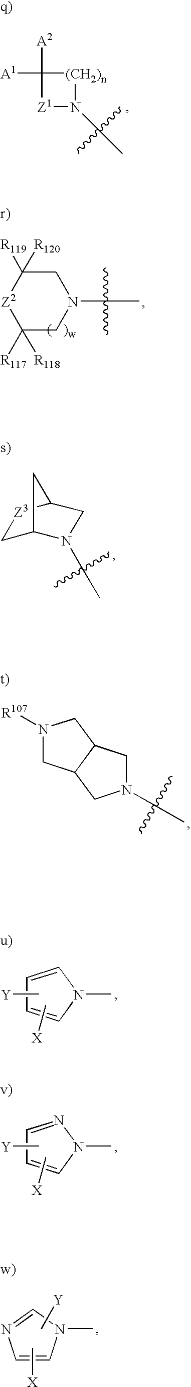 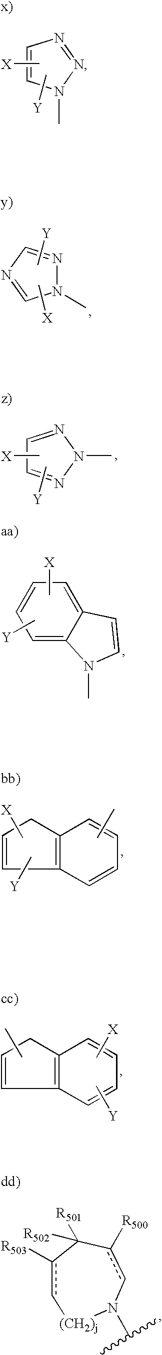 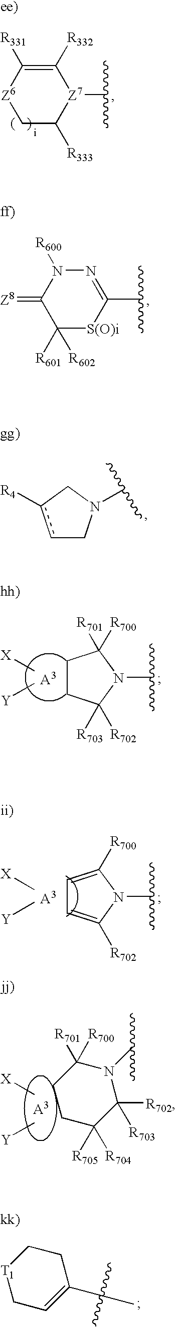 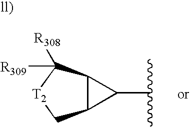
B and one R₂ are taken together to form 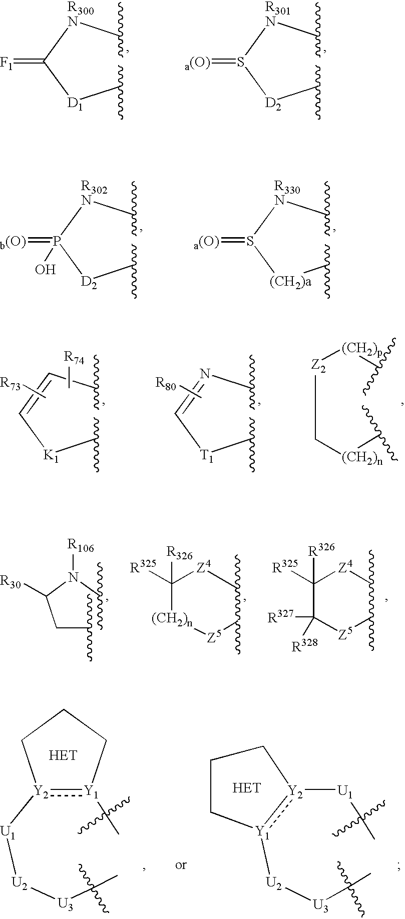
A¹ is
   a) H—, or
   b) CH₃;
A² is
   a) H—,
   b) HO—,
   c) CH₃—,
   d) CH₃O—,
   e) R¹⁰²O—CH₂—C(O)—NH—
   f) R¹⁰³O—C(O)—NH—,
   g) (C₁-C₂)alkyl-O—C(O)—,
   h) HO—CH₂—,
   i) CH₃O—NH—,
   j) (C₁-C₃)alkyl-O₂C—
   k) CH₃—C(O)—,
   l) CH₃—C(O)—CH₂—, 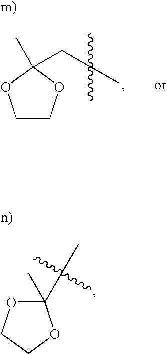
A¹ and A² taken together are: 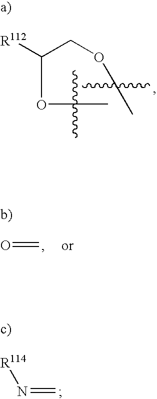
A³ represents any 5-10 membered aryl ring or aromatic het, the het having 1-4 heteroatoms selected from O, S, or N;
B₁ is
   a) —N═C(H)—C(H)═C(H)—, or
   b) —C(H)═N—C(H)═C(H)—;
D₁ is
   a) O,
   b) S(O)ᵢ, or
   c) —N(R₃₀₄)—;
D₂ is
   a) O, or
   b) —N(R₃₀₄)—;
E is
   a) NR₃₉,
   b) —S(═O)ᵢ,
   c) O, or
   d) —S(═O)(═NR₃₁₅);
F₁ is
   a) O,
   b) S,
   c) NH,
   d) N—OH,
   e) N—O—C₁₋₄ alkyl,
   f) N—OC(O)—C₁₋₄ alkyl, or
   g) N—C₁₋₄alkyl;
K₁ is
   a) O,
   b) S, or
   c) —NR₃₀₅—;
M is
   a) H,
   b) C₁₋₈ alkyl,
   c) C₃₋₈ cycloalkyl,
   d) —(CH₂)ₘOR₁₃, or
   e) —(CH₂)ₕ—NR₂₁R₂₂;
T₁ is
   a) —O—,
   b) —NR₃₀₆—
   c) —S(O)ᵢ—,
   d) —C(R₂)—, or
   e) —C(O)—;
T₂ is
   a) —O—,
   b) —NR₃₀₇—,
   c) —S(O)ᵢ—,
   d) —C(O)—, or
   e) —C(R₂)₂—;
Each U₁, U₂, and U₃ is independently selected from
   a) —C(R₁)₂—,
   b) —NR₃₉—,
   c) —O—, or
   d) —S(O)ᵢ—;
V is
   a) O,
   b) CH₂, or
   c) NR₈₇;
W is
   a) CH, or
   b) N;
W₁ is
   a) —NH—,
   b) O, or
   c) S;
W₂ is O or S;
X is
   a) H,
   b) —CN,
   c) —OR₂₇,
   d) halo,
   e) —NO₂,
   f) tetrazolyl optionally substituted with C₁₋₄alkyl,
   g) —SH,
   h) —S(═O)ᵢR₄,
   i) —SC(═O)R₇,
   j) —C(═O)R₂₅,
   k) —C(═O)NR₂₇R₂₈,
   l) —C(═NR₂₉)R₂₅,
   m) —C(R₂₅)(R₂₈)—OR₁₃,
   n) —C(R₂₅)(R₂₈)—OC(═O)R₁₃,
   o) —C(R₂₈)(OR₁₃)—(CH₂)ₕ—NR₂₇R₂₈,
   p) —NR₂₇R₂₈,
   q) —N(R₂₇)C(═O)R₇,
   r) —N(R₂₇)—S(═O)ᵢR₇,
   s) —C(OR₁₄)(OR₁₅)R₂₈,
   t) —C(R₂₅)(R₁₆)—NR₂₇R₂₆,
   u) —C₁₋₈ alkyl substituted with one or more halos, OH, ═O other than at alpha position, —S(═O)ᵢR₁₇, —NR₂₇R₂₈, C₂₋₅ alkenyl, C₂₋₅ alkynyl, or C₃₋₈ cycloalkyl, or
   v) -Het optionally substituted with R₂, ═O or ═S;
Y is
   a) H,
   b) F,
   c) Cl,
   d) Br,
   e) C₁₋₃ alkyl, or
   f) NO₂;
Each Y₁ and Y₂ is independently
   a) CH,
   b) N, provided that . . . is absent, or
   c) C when . . . is present;
Z is
   a) O,
   b) S, or
   c) NM;
Z₁ is
   a) —CH₂—,
   b) —CH(R¹⁰⁴)—CH₂—,
   c) —C(O)—, or
   d) —CH₂CH₂CH₂—;
Z² is
   a) —S(O)ᵢ—,
   b) —O—,
   c) —N(R¹⁰⁷)—, or
   d) —S(═O)(═NR³¹⁵)—;
Z³ is
   a) —S(O)i-, or
   b) —O—,
Z⁴ is
   a) —S(═O)ᵢ—, or
   b) —NR³⁰³—;
Z⁵ is
   a) —O—,
   b) —NH—,
   c) —CH₂—,
   d) —C(halo)₂-, or
   e) —S(═O)ᵢ—;
Z⁶ is
   a) S(═O)ᵢ,
   b) S(═NR³¹⁵), or
   c) S(═NR³¹⁵)(═O);
Z⁷ is
   a) N,
   b) CR¹¹⁰,
   c) CR¹¹⁵, or
   d) CR¹¹⁶;
Z⁸ is
   a) O, or
   b) S;
R₁ is
   a) H,
   b) —OH,
   c) C₁₋₆ alkyl optionally substituted with one or more halos, —OH, —CN, aryl, het, alkoxy, substituted aryl or substituted het,
   d) C₁₋₆ alkoxy optionally substituted with one or more halos, —OH, —CN, aryl, het, substituted aryl or substituted het,
   e) C₂₋₆ alkenyl optionally substituted with aryl, het, substituted aryl or substituted het,
   f) —NH₂, or
   g) C₃₋₅ cycloalkyl;
R₂ is
   a) H,
   b) C₁₋₂ alkyl optionally substituted with one or more halos,
   c) —NH₂,
   d) —NO₂,
   e) —CN, or
   f) halo;
R₄ is
   a) H
   b) C₁₋₄ alkyl optionally substituted with one or more halos, OH, CN, NR₁₀R₁₁, or —CO₂R₁₃,
   c) C₂₋₄ alkenyl,
   d) —NR₁₆R₁₈,
   e) —NHC(═O)R₇,
   f) —NR₂₀C(═O)R₇,
   g) —N(R₁₇)₂,
   h) —NR₁₆R₁₇, or
   i) —NR₁₇R₂₀;
R₅ and R₆ at each occurrence are the same or different and are
   a) C₁₋₂ alkyl, or
   b) R₅ and R₆ taken together are —(CH₂)ₖ—;
R₇ is
   a) C₁₋₄ alkyl optionally substituted with one or more halos;
R₁₀ and R₁₁ at each occurrence are the same or different and are
   a) H,
   b) C₁₋₄ alkyl, or
   c) C₃₋₈ cycloalkyl;
R₁₃ is
   a) H, or
   b) C₁₋₄ alkyl;
R₁₄ and R₁₅ at each occurrence are the same or different and are
   a) C₁₋₄ alkyl, or
   b) R₁₄ and R₁₅ taken together are —(CH₂)₁—;
R₁₆ is
   a) H,
   b) C₁₋₄ alkyl, or
   c) C₃₋₈ cycloalkyl;
R₁₇ is
   a) C₁₋₄ alkyl, or
   b) C₃₋₈ cycloalkyl;
R₁₈ is
   a) H,
   b) C₁₋₄ alkyl,
   c) C₂₋₄ alkenyl,
   d) C₃₋₄ cycloalkyl,
   e) —OR₁₃ or
   f) —NR₂₁R₂₂;
R₂₀ is a physiologically acceptable cation, such as sodium, potassium, lithium, calcium or magnesium;
R₂₁ and R₂₂ at each occurrence are the same or different and are
   a) H,
   b) C₁₋₄ alkyl, or
   c) R₂₁ and R₂₂ taken together are —(CH₂)ₘ—;
R₂₅ is
   a) H,
   b) C₁₋₈ alkyl optionally substituted with one or more halos, C₃₋₈ cycloalkyl, C₁₋₄ alkyl substituted with one or more of —S(═O)ᵢR₁₇, —OR₁₃, or OC(═O)R₁₃, NR₂₇R₂₈, or
   c) C₂₋₅ alkenyl optionally substituted with —C(O)H, or CO₂R₁₃;
R₂₆ is
   a) R₂₈, or
   b) —NR₂₇N₂₈;
R₂₇ and R₂₈ at each occurrence are the same or different and are
   a) H,
   b) C₁₋₈ alkyl,
   c) C₃₋₈ cycloalkyl,
   d) —(CH₂)ₘOR₁₃,
   e) —(CH₂)ₕ—NR₂₁R₂₂, or
   f) R₂₇ and R₂₈ taken together are —(CH₂)₂O(CH₂)₂—, —(CH₂)ₕCH(COR₇)—, or —(CH₂)₂N(CH₂)₂(R₇);
R₂₉ is
   a) —NR₂₇R₂₈,
   b) —OR₂₇, or
   c) —NHC(═O)R₂₈;
R₃₀ is
   a) H, or
   b) C₁₋₄ alkyl optionally substituted with one or more halos, OH, C₁₋₄ alkoxy, CN, SH, NH₂, —OR₃₁, —NHR₃₁, —N(R₃₁)₂, or —S(O)iR₃₁;
R₃₁ is
   a) C₁₋₄ alkyl,
   b) —C(O)C₁₋₄ alkyl,
   c) —C(O)OC₁₋₄ alkyl,
   d) —C(O)NH₂,
   e) —C(O)NHC₁₋₄ alkyl, or
   f) —SO₂C₁₋₄ alkyl;
R₃₈ is
   a) H,
   b) C₁₋₆ alkyl,
   c) —(CH₂)_{q}-aryl, or
   d) halo;
R₃₉ is
   a) H,
   b) C₁₋₆ alkyl optionally substituted with one or more OH, halo, or —CN,
   c) —(CH₂)_{w}-aryl,
   d) —CO₂R₄₀,
   e) —COR₄₁,
   f) —C(═O)—(CH₂)_{q}—C(═O)R₄₀,
g) —S(═O)₂—C₁₋₆ alkyl,
   h) —S(═O)₂—(CH₂)_{q}-aryl, or
   i) —(C═O)ⱼ-Het;
R₄₀ is
   a) H,
   b) C₁₋₆ alkyl optionally substituted with one or more OH, halo, or —CN,
   c) —(CH₂)_{q}-aryl, or
   d) —(CH₂)_{q}—OR₄₂;
R₄₁ is
   a) C₁₋₆ alkyl optionally substituted with one or more OH, halo, —OP(O)(OH)₂, —OP(OH)₂, or —CN,
   b) —(CH₂)_{q}-aryl, or
   c) —(CH₂)_{q}—OR₄₂;
R₄₂ is
a) H,
b) C₁₋₆ alkyl,
c) —(CH₂)_{q}-aryl, or
   d) —C(═O)—C₁₋₆ alkyl;
R₄₉ and R₅₀ at each occurrence are the same or different and are
   a) H,
   b) C₁₋₄ alkyl,
   c) C₅₋₆ cycloalkyl, or
   d) R₄₉ and R₅₀ taken together with the nitrogen atom is a 5-, 6-membered saturated heterocyclic moiety which optionally has a further hetero atom selected from the group consisting of S, N, and O, and can in turn be optionally substituted with, including on the further nitrogen atom, C₁₋₃ alkyl, or C₁₋₃ acyl;
R₅₁ is
   a) carboxyl,
   b) halo,
   c) —CN,
   d) mercapto,
   e) formyl,
   f) CF₃,
   g) —NO₂,
   h) C₁₋₆ alkoxy,
   i) C₁₋₆ alkoxycarbonyl,
   j) C₁₋₆ alkythio,
   k) C₁₋₆ acyl,
   l) C₁₋₆ alkyl optionally substituted with OH, C₁₋₅ alkoxy, C₁₋₅ acyl, or —NR₄₉R₅₀,
   m) phenyl,
   n) —C(═O)NR₅₂R₅₃,
   o) —NR₄₉R₅₀,
   p) —N(R₅₂)(—SO₂R₅₄),
   q) —SO₂—NR₅₂R₅₃, or
   r) —S(═O)ᵢR₅₄;
R₅₂ and R₅₃ at each occurrence are the same or different and are
   a) H,
   b) C₁₋₆ alkyl, or
   c) phenyl;
R₅₄ is
   a) C₁₋₄ alkyl, or
   b) phenyl optionally substituted with C₁₋₄ alkyl;
R₇₃ and R₇₄ at each occurrence are the same or different and are
   a) H,
   b) carboxyl,
   c) halo,
   d) —CN,
   e) mercapto,
   f) formyl,
   g) CF₃,
   h) —NO₂,
   i) C₁₋₆ alkoxy,
   j) C₁₋₆ alkoxycarbonyl,
   k) C₁₋₆ alkythio,
   l) C₁₋₆ acyl,
   m) —NR₇₈R₇₉,
   n) C₁₋₆ alkyl optionally substituted with OH, C₁₋₅ alkoxy, C₁₋₅ acyl, —NR₇₈R₇₉, —N(phenyl)(CH₂—CH₂—OH), —O—CH(CH₃)(OCH₂CH₃), or —O-phenyl-[para-NHC(═O)CH₃],
   o) C₂₋₈ alkenylphenyl optionally substituted with R₅₁,
   p) phenyl optionally substituted with R₅₁, or
   q) a 5-, or 6-membered saturated or unsaturated heterocyclic moiety having one to three atoms selected from the group consisting of S, N, and O, optionally substituted with R₅₁;
R₇₈ and R₇₉ at each occurrence are the same or different and are
   a) H,
   b) C₁₋₄ alkyl,
   c) phenyl, or
   d) R₇₈ and R₇₉ taken together with the nitrogen atom is a 5-, 6-membered saturated heterocyclic moiety which optionally has a further hetero atom selected from the group consisting of S, N, and O, and can in turn be optionally substituted with, including on the further nitrogen atom, C₁₋₃ alkyl, or C₁₋₃ acyl;
R₈₀ is
   a) H,
   b) formyl,
   c) carboxyl,
   d) C₁₋₆ alkoxycarbonyl,
   e) C₁₋₈ alkyl,
   f) C₂₋₈ alkenyl,
      wherein the substituents (e) and (f) can be optionally substituted with OH, halo, C₁₋₆ alkoxy, C₁₋₆ acyl, C₁₋₆ alkylthio or C₁₋₆ alkoxycarbonyl, or phenyl optionally substituted with halo,
   g) an aromatic moiety having 6 to 10 carbon atoms optionally substituted with carboxyl, halo, —CN, formyl, CF₃, —NO₂, C₁₋₆ alkyl, C₁₋₆ alkoxy, C₁₋₆ acyl, C₁₋₆ alkylthio, or C₁₋₆ alkoxycarbonyl;
   h) —NR₈₁R₈₂,
   i) —OR₉₀,
   j) —S(═O)ᵢ—R₉₁, or
   k) —SO₂—N(R₉₂)(R₉₃);
R₈₁ and R₈₂ at each occurrence are the same or different and are
   a) H,
   b) C₃₋₆ cycloalkyl,
   c) phenyl,
   d) C₁₋₆ acyl,
   e) C₁₋₈ alkyl optionally substituted with OH, C₁₋₆ alkoxy which can be substituted with OH, a 5-, or 6-membered aromatic heterocyclic moiety having one to three atoms selected from the group consisting of S, N, and O, phenyl optionally substituted with OH, CF₃, halo, —NO₂, C₁₋₄ alkoxy, —NR₈₃R₈₄, or 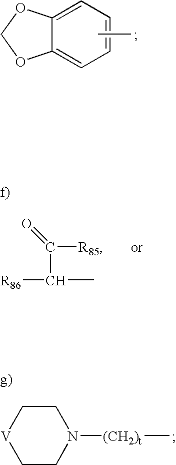
R₈₃ and R₈₄ at each occurrence are the same or different and are
   a) H, or
   b) C₁₋₄ alkyl;
R₈₅ is
   a) OH,
   b) C₁₋₄ alkoxy, or
   c) —NR₈₈R₈₉;
R₈₆ is
   a) H, or
   b) C₁₋₇ alkyl optionally substituted with indolyl, OH, mercaptyl, imidazoly, methylthio, amino, phenyl optionally substituted with OH, —C(═O)—NH₂, —CO₂H, or —C(═NH)—NH₂;
R₈₇ is
   a) H,
   b) phenyl, or
   c) C₁₋₆ alkyl optionally substituted by OH;
R₈₈ and R₈₉ at each occurrence are the same or different and are
   a) H,
   b) C₁₋₅ alkyl
   c) C₃₋₆ cycloalkyl, or
   d) phenyl;
R₉₀ is
   a) C₁₋₈ alkyl optionally substituted with C₁₋₆ alkoxy or C₁₋₆ hydroxy, C₃₋₆ cycloalkyl, a 6-membered aromatic optionally benzo-fused heterocyclic moiety having one to three nitrogen atoms, which can in turn be substituted with one or two —NO₂, CF₃, halo, —CN, OH, C₁₋₅ alkyl, C₁₋₅ alkoxy, or C₁₋₅ acyl; 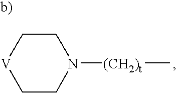
   c) phenyl, or
   d) pyridyl;
R₉₁ is
   a) C₁₋₆ alkyl,
   b) C₂₋₁₆ alkenyl,
      wherein the substituents (a) and (b) can be optionally substituted with C₁₋₆ alkoxycarbonyl, or a 5-, 6-, 7-membered aromatic heterocyclic moiety having one to three atoms selected from the group consisting of S, N, and O,
   c) an aryl having 6 to 10 carbon atoms, or
   d) a 5-, 6-, 7-membered aromatic heterocyclic moiety having one to three atoms selected from the group consisting of S, N, and O, wherein the substituents (c) and (d) can be optionally substituted with carboxyl, halo, —CN, formyl, CF₃, —NO₂, C₁₋₆ alkyl, C₁₋₆ alkoxy, C₁₋₆ acyl, C₁₋₆ alkylthio, or C₁₋₆ alkoxycarbonyl;
R₉₂ and R₉₃ at each occurrence are the same or different and are
   a) H,
   b) phenyl,
   c) C₁₋₆ alkyl, or
   d) benzyl;
R¹⁰² is
   a) H—,
   b) CH₃—,
   c) phenyl-CH₂—, or
   d) CH₃C(O)—;
R¹⁰³ is
   a) (C₁-C₃)alkyl-, or
   b) phenyl-;
R¹⁰⁴ is
   a) H—, or
   b) HO—;
R¹⁰⁶ is
   a) CH₃—C(O)—,
   b) H—C(O)—,
   c) Cl₂CH—C(O)—,
   d) HOCH₂—C(O)—,
   e) CH₃SO₂—, 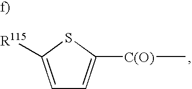
   g) F₂CHC(O)—, 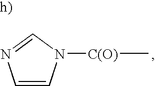
   i) H₃C—C(O)—O—CH₂—C(O)—,
   j) H—C(O)—O—CH₂—C(O)—, 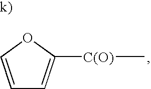
   l) HC≡—C—CH₂O—CH₂—C(O)—,
   m) phenyl-CH₂—O—CH₂—C(O)—,
   o) C₁₋₄alkyl-NH—C(S)—, or
   p) C₁₋₄alkyl optionally substituted with one or more halo, CN, NO₂, OH, SH, or NH₂;
R¹⁰⁷ is
   a) R¹⁰²O—C(R¹¹⁰)(R¹¹¹)—C(O)—,
   b) R¹⁰³O—C(O)—,
   c) R¹⁰⁸—C(O)—, 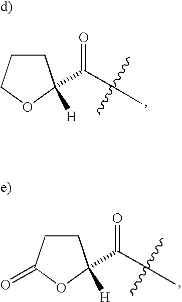
   f) H₃C—C(O)—(CH₂)₂—C(O)—,
   g) R¹⁰⁹—SO₂—, 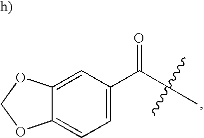
   i) HO—CH₂—C(O)—,
   j) R¹¹⁶—(CH₂)₂—,
   k) R¹¹³—C(O)—O—CH₂—C(O)—,
   l) (CH₃)₂N—CH₂—C(O)—NH—,
   m) NC—CH₂—,
   n) F₂—CH—CH₂—, or
   o) R¹⁵⁰R¹⁵¹NSO₂
R¹⁰⁸ is
   a) H—,
   b) (C₁-C₄)alkyl,
   c) aryl-(CH₂)ₙ,
   d) ClH₂C—,
   e) Cl₂HC—,
   f) FH₂C—,
   g) F₂HC—,
   h) (C₃-C₆)cycloalkyl, or
   i) CNCH₂—.
R¹⁰⁹ is
   a) C₁-C₄alkyl,
   b) —CH₂Cl
   c) —CH₂CH═CH₂,
   d) aryl, or
   e) —CH₂CN;
R¹¹⁰ and R¹¹¹ are independently
   a) H—,
   b) CH₃—; or
R¹¹² is
   a) H—,
   b) CH₃O—CH₂O—CH₂—, or
   c) HOCH₂—;
R¹¹³ is
   a) CH₃—,
   b) HOCH₂—,
   c) (CH₃)₂N-phenyl, or
   d) (CH₃)₂N—CH₂—;
R¹¹⁴ is
   a) HO—,
   b) CH₃O—,
   c) H₂N—,
   d) CH₃O—C(O)—O—,
   e) CH₃—C(O)—O—CH₂—C(O)—O—,
   f) phenyl-CH₂—O—CH₂—C(O)—O—,
   g) HO—(CH₂)₂—O—,
   h) CH₃O—CH₂—O—(CH₂)₂—O—, or
   i) CH₃O—CH₂—O—;
R¹¹⁵ is
   a) H—, or
   b) Cl—;
R¹¹⁶ is
   a) HO—
   b) CH₃O—, or
   c) F;
Each of R₁₁₇, R₁₁₈, R₁₁₉, and R₁₂₀ is independently selected from
   a) H,
   b) C₁-C₆alkyl,
   c) substituted alkyl,
   d) halo, or
   e) R₁₁₇ and R₁₁₈ or R₁₁₉ and R₁₂₀ together are ═O;
R¹⁵⁰ and R¹⁵¹ are each independently
   a) H,
   b) C₁-C₄alkyl, or
   c) R¹⁵⁰ and R¹⁵¹ taken together with the nitrogen atom, to which R¹⁵⁰ and R¹⁵¹ are attached, form a monocyclic heterocyclic ring having from 3 to 6 carbon atoms;
Each R₃₀₀, R₃₀₁, R₃₀₂, R₃₀₃, R₃₀₄, R₃₀₅, and R₃₀₆ is independently selected from
   a) H,
   b) C₃₋₆ cycloalkyl optionally substituted with ═O,
   c) C₁₋₆ alkoxy,
   d) C₁₋₁₀ alkyl optionally substituted with one or more of R₃₁₀,
   e) C₂₋₁₀ alkenyl optionally substituted with one or more of R₃₁₀,
   f) benzyloxycarbonyl,
   g) aryl,
   h) het,
   i) —C(O)—NR₃₁₁R₃₁₂,
   j) —S(O)₂—NR₃₁₁R₃₁₂,
   k) —(O)ᵢSR₃₁₁,
   l) —C(O)—R₃₁₀,
   m) —C(S)—NR₃₁₁R₃₁₂,
   n) —C(O)—H, or
   o) —C(O)—C₁₋₄alkyl optionally substituted with one or more of R₃₁₀;
Each R₃₀₇ is independently selected from
   a) H,
   b) C₃₋₆ cycloalkyl optionally substituted with ═O,
   c) C₁₋₆ alkoxy,
   d) C₁₋₁₀ alkyl optionally substituted with one or more of R₃₁₀,
   e) C₂₋₁₀ alkenyl optionally substituted with one or more of R₃₁₀,
   f) benzyloxycarbonyl,
   g) aryl,
   h) het,
   i) —C(O)—NR₃₁₁R₃₁₂,
   j) —S(O)₂—NR₃₁₁R₃₁₂,
   k) —(O)ᵢSR₃₁₁,
   l) —C(O)—R₃₁₀,
   m) —C(S)—NR₃₁₁R₃₁₂,
   n) —C(O)—H,
   o) —C(O)—C₁₋₄alkyl optionally substituted with one or more of R₃₁₀, or
   p) —C(N—O—C₁₋₄alkyl)-;
R₃₀₈ and R₃₀₉ are H or both R₃₀₈ and R₃₀₉ together form ═O or ═S;
R₃₁₀ is
   a) —CN,
   b) —N₃,
   c) —CF₃,
   d) pyridyl,
   e) halo,
   f) —OH,
   g) —O(O)C₁-C₆alkyl,
   h) —C₁₋₆ alkyloxycarbonyl,
   i) —SH,
   j) —NH₂;
Each R₃₁₁ and R₃₁₂ is independently selected from
   a) H,
   b) C₁₋₄ alkyl,
   c) phenyl, or
   d) R₃₁₁ and R₃₁₂ together with the N-atom to which they are attached forms a 5- or 6-membered, saturated heterocyclic ring optionally having one or more O, S, or N atoms in the ring, the heterocyclic ring being optionally substituted with C₁₋₃ alkyl;
R₃₁₅ is
   a) H,
   b) C₁₋₄ alkyl optionally substituted with halo, —OH, C₁₋₈ alkoxy, amino, C₁₋₈ alkylamino, or C₁₋₈dialkylamino,
   c) aryl-S(O)₂—,
   d) C(═O)C₁₋₄alkyl,
   e) C(═O)OC₁₋₄alkyl,
   f) C(═O)NHR₃₂₀,
   g) C(═S)NHR₃₂₀,
   h) —OC(═O)C₁₋₄alkyl,
   i) —S(O)ᵢC₁₋₄alkyl,
   j) C₁₋₄ alkyl-O—C₁₋₄ alkyl, or
   k) C₁₋₄ alkyl-S—C₁₋₄ alkyl;
R₃₂₀ is independently selected from
   a) H, or
   b) substituted alkyl;
Each R₃₂₅, R₃₂₆, R₃₂₇, and R₃₂₈ is independently selected from
   a) H,
   b) C₁-C₆alkyl,
   c) substituted alkyl,
   d) halo, or
   e) R₃₂₅ and R₃₂₆ or R₃₂₇ and R₃₂₈ together are ═O, ═S, or ═N—R₃₃₂, or
   f) one of R₃₂₅ or R₃₂₆ and R₃₀₃, when Z₄ is —N(R₃₀₃)—, together with the carbon and nitrogen atoms to which they are bound form a 5-7 membered het containing one or more heteroatoms selected from O, S, or N, the het being optionally substituted with one or more of R₂, ═O, or ═S;
R₃₃₀ is
   a) H, or
   b) alkyl, or
   c) substituted alkyl;
R₃₃₁ is
   a) R₃₃₂,
   b) Cl,
   c) NH₂,
   d) OH,
   e) NHC₁-C₄alkyl, or
   f) R₃₁₅;
R₃₃₂ is
   a) H,
   b) C₁-C₄alkyl,
   c) OC₁-C₄alkyl,
   d) SC₁-C₄alkyl, or
   e) NHC₁-C₄alkyl;
R₃₃₃ is
   a) F, or
   b) R₃₃₂;
R₅₀₀ and R₅₀₃ are each and independently
   (a) H,
   (b) halo,
   (c) C₁-C₈ alkyl,
   (d) C₃-C₆ cycloalkyl,
   (e) —(CH₂)ᵢ—OR₅₁₁, or
   (f) —C(═O)—R₅₄₁;
R₅₀₁ and R₅₀₂ are each and independently
   (a) hydrogen atom,
   (b) C₁-C₈ alkyl,
   (c) C₁-C₈ alkoxy,
   (d) C₁-C₈ alkylthio,
   (e) —(CH₂)ᵢ—OR₅₅₁,
   (f) —O—(CH₂)ᵢ—OR₅₅₁,
   (g) —NR₅₄₂R₅₅₂,
   (h) —C(═O)—NR₅₄₂R₅₅₂,
   (i) —(CH₂)ᵢ—C(═O)—R₅₄₁,
or R₅₀₁ and R₅₀₂ together form
   (j) ═O,
   (k) ═NR₅₄₃,
   (l) ═S,
   (m) ═CR₅₄₄R₅₅₄, or
   (n) an unsaturated or saturated 5- or 6-membered hetero ring having 1-3 hetero atoms selected from the group consisting of a nitrogen atom, an oxygen atom and a sulfur atom;
R₅₁₁ and R₅₁₂ are each and independently
   (a) hydrogen atom,
   (b) C₁-C₈ alkyl;
R₅₄₁ is
   (a) hydrogen atom,
   (b) —(CH₂)ₘ—OH,
   (c) C₁-C₈ alkyl,
   (d) C₁-C₈ alkoxy, or
   (e) —O—CH₂—O—C(═O)—R₅₁₁;
R₅₄₂ and R₅₅₂ are each and independently
   (a) hydrogen atom,
   (b) —(CH₂)ᵢ—OH,
   (c) C₁-C₈ alkyl,
   (d) —C(═O)—R₅₄₁,
   (e) —C(═O)—NR₅₁₁R₅₁₂,
   (f) —(CH₂)_{q}-phenyl, or
or R₅₄₂ and R₅₅₂ together form a pyrrolidino group, a piperidino group, a piperazino group, a morpholino group, or a thiomorpholino group, each of which may be substituted by C₁-C₈ alkyl or —(CH₂)ᵢ—OH;
R₅₄₃ is
   (a) H,
   (b) —OR₅₅₁,
   (c) C₁-C₈ alkyl,
   (d) C₁-C₈ alkoxy,
   (e) —(CH₂)_{q}-phenyl,
   (f) —NR₅₄₂R₅₅₂,
   (g) —NH—C(═NH)—NH₂, or
   (h) [1,2,4]triazol-4-yl;
R₅₄₄ and R₅₅₄ are each and independently
   (a) H,
   (b) C₁-C₈ alkyl,
   (c) —C(═O)—R₅₄₁, or
   (d) —(CH₂)_{q}-phenyl;
R₅₅₁ is
   (a) H,
   (b) C₁-C₈ alkyl,
   (c) C₁-C₈ alkyl substituted with 1-3 halo,
   (d) —(CH₂)ᵢ—OR₅₁₁,
   (e) —(CH₂)ᵢ—C(═O)—R₅₄₁, or
   (f) —C(═O)—(CH₂)ᵢ—OR₅₄₄;
R₆₀₀ is
   a) H,
   b) C₁-C₄alkyl
   c) het,
   d) (CH₂)_{b}C(O)OC₁-C₄alkyl,
   e) (CH₂)_{b}C(O)C₁-C₄alkyl, or
   f) aryl;
R₆₀₁ and R₆₀₂ are each independently
   a) H,
   b) C₁-C₄alkyl,
   c) het,
   d) C₃-C₆cycloalkyl,
   e) aryl,
   f) OC₁-C₄alkyl,
   g) C(O)OC₁-C₄alkyl; or
   h) R₆₀₁ and R₆₀₂ taken together along with the carbon atom to which they attach form a C₃-C₆cycloalkyl;
Each R₇₀₀, R₇₀₁, R₇₀₂, R₇₀₃, R₇₀₄, and R₇₀₅ is independently selected from
   a) H,
   b) C₁₋₄ alkyl optionally substituted with 1-3 halo, ═O, ═S, —OH
   c) C(O)NH₂,
   d) —CN,
   e) aryl,
   f) substituted aryl,
   g) het,
   h) substituted het,
   i) C(O)OH,
   j) C(O)OC₁₋₄ alkyl, or
   k) R₇₀₀ and R₇₀₁ form ═O or ═S, or
   l) R₇₀₂ and R₇₀₃ form ═O or ═S, or
   m) R₇₀₄ and R₇₀₅ form ═O or ═S;
a is 1 or 2;
b is 0 or i;
h is 1, 2, or 3;
i is 0, 1, or 2;
j is 0 or 1;
k is 3, 4, or 5;
l is 2 or 3;
m is 2, 3, 4 or 5;
n is 0, 1, 2, 3, 4, or 5;
p is 0, 1, 2, 3, 4, or 5; with the proviso that n and p together are 1, 2, 3, 4, or 5;
q is 1, 2, 3, or 4;
t is 0, 1, 2, 3, 4, 5, or 6; and
w is 0, 1, 2, or 3.

Embodiments of this aspect of the invention may include one or more of the following features. 21. R₂ is independently selected from H, F, Cl, Br, CN, NH₂, NO₂, CF₃, and CH₃. The structure of A is 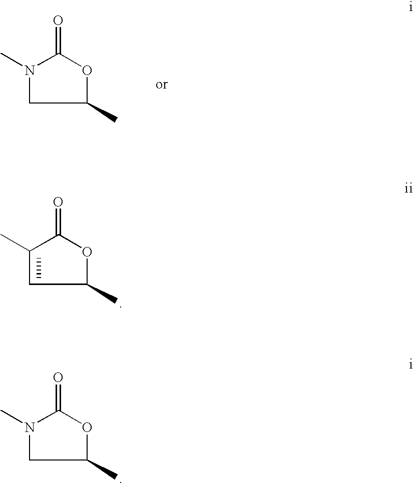
The structure of A is R₁ is H, —NH₂, —OH, C₁₋₄alkyl, C₃₋₅ cycloalkyl, C₁₋₄ alkoxy, or C₂₋₄ alkenyl, the alkyl and alkoxy each optionally being substituted with one or more halo, —OH, —CN. R₁ is H, —OH, —CH₂—CH═CH₂, methyl, ethyl, propyl, —CH₂—CH₂F, —CH₂—CH₂OH, or methoxy. B is 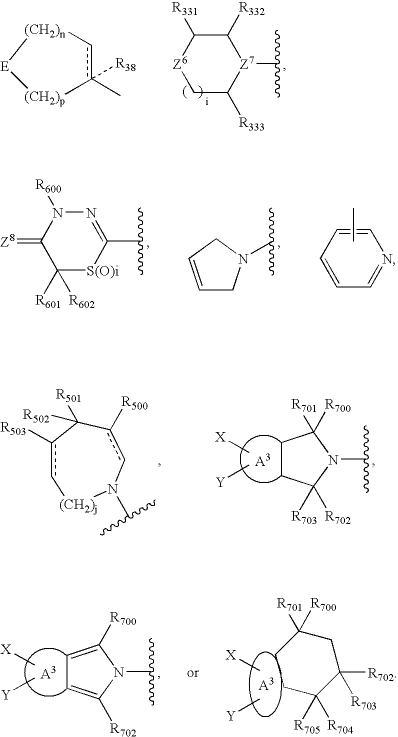
B is selected from 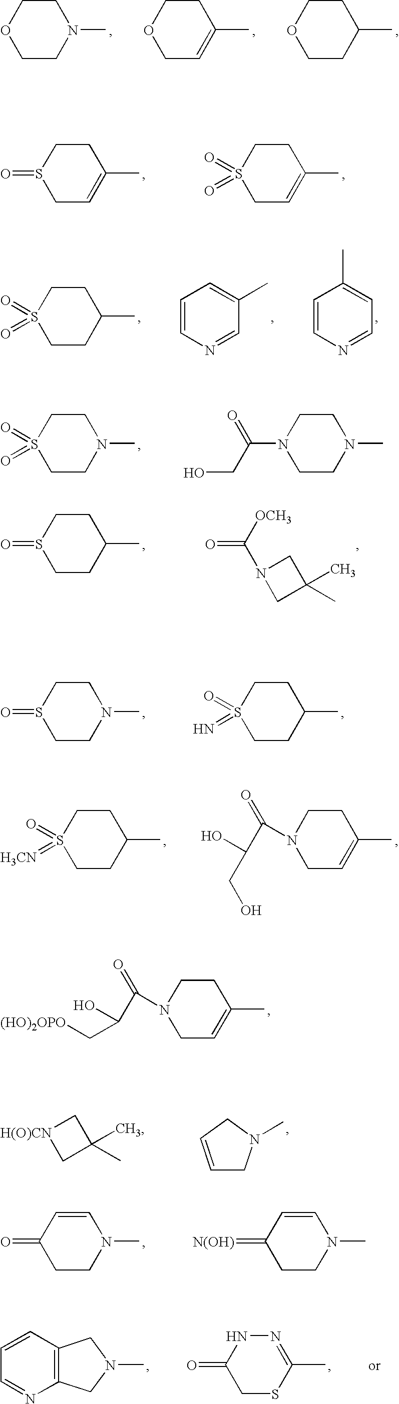 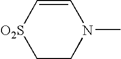
B and one R₂ form 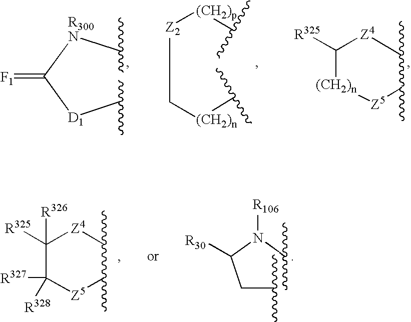
B and one R₂ form —S—C(O)—N(R₃₀₀)—, —O—C(O)—N(R₃₀₀)—, —N(R₁₀₆)—HCR₃₀—CH₂—, —NR₃₀₀—C(O)—C(R₃₂₇R₃₂₈)—O—, —NR₃₀₀—C(O)—C(R₃₂₇R₃₂₈)—S—, —NR₃₀₀—C(S)—C(R₃₂₇R₃₂₈)—S—, —NR₃₀₀—C(O)—C(R₃₂₇R₃₂₈)—CH₂—, —CH₂—CH₂—NR₁₀₇—CH₂—CH₂—, or —CH₂—NR₁₀₇—CH₂—CH₂—CH₂—. D₁ is S. D₁ is O. R₃₀₀ is C₁₋₄ alkyl such as methyl, ethyl, or isopropyl. One R₂ is hydrogen and the other R₂ is F. Both R₂ substituents are F.

Other aspects of the invention include pharmaceutical compositions including a compound of formulae I, II, or III and a pharmaceutically acceptable carrier, and methods for treating microbial infections in mammals by administering an effective amount of a compound of formulae I, II, or III. The compound may be administered to the mammal orally, parenterally, transdermally, or topically in a pharmaceutical composition. The compound may be administered in an amount of from about 0.1 to about 100 mg/kg of body weight/day, such as in an amount of from about 1 to about 50 mg/kg of body weight/day.

### DETAILED DESCRIPTION OF THE INVENTION

The following definitions are used, unless otherwise described.

Variables including a letter followed by a numeric superscript are equivalent to the same letter followed by the same number as a subscript. For instance, R¹ and R₁ are equivalent and refer to the same variable.

The carbon atom content of various hydrocarbon-containing moieties may be indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₇ alkyl refers to alkyl of one to seven carbon atoms, inclusive.

The term “halo” refers to a halogen atom selected from Cl, Br, I, and F.

The term “alkyl” refers to both straight- and branched-chain moieties. Unless otherwise specifically stated, such as by a Cᵢ₋ⱼ prefix, alkyl moieties include between 1 and 6 carbon atoms.

The term “alkenyl” refers to both straight- and branched-chain moieties containing at least one —C═C—. Unless otherwise specifically stated, such as by a Cᵢ₋ⱼ prefix, alkenyl moieties include between 2 and 6 carbon atoms.

The term “alkynyl” refers to both straight- and branched-chain moieties containing at least one —C≡C—. Unless otherwise specifically stated, such as by a Cᵢ₋ⱼ prefix, alkynyl moieties include between 2 and 6 carbon atoms.

The term “alkoxy” refers to —O-alkyl groups. Unless otherwise specifically stated, such as by a Cᵢ₋ⱼ prefix, the alkyl portion of the —O-alkyl group includes between 1 and 6 carbon atoms.

The term “amino” refers to NH₂.

The term “cycloalkyl” refers to a cyclic alkyl moiety. Unless otherwise specifically stated, such as by a Cᵢ₋ⱼ prefix, cycloalkyl moieties will include between 3 and 7 carbon atoms.

The term “cycloalkenyl” refers to a cyclic alkenyl moiety. Unless otherwise specifically stated, such as by a Cᵢ₋ⱼ prefix, cycloalkenyl moieties will include between 3 and 7 carbon atoms and at least one —C═C— group within the cyclic ring.

The term “aryl” refers to phenyl and naphthyl.

The term “het” refers to mono- or bicyclic ring systems containing at least one heteroatom selected from O, S, and N. Each monocyclic ring may be aromatic, saturated, or partially unsaturated. A bicyclic ring system may include a monocyclic ring containing at least one heteroatom which is fused with a cycloalkyl or aryl group. A bicyclic ring system may also include a monocyclic ring containing at least one heteroatom fused with another het, monocyclic ring system.

Examples of “het” include, but are not limited to, pyridine, thiophene, furan, pyrazoline, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl, 5-isothiazolyl, 1,3,4,-oxadiazole, 4-oxo-2-thiazolinyl, 5-methyl-1,3,4-thiadiazol-2-yl, thiazoledione, 1,2,3,4-thiatriazole, 1,2,4-dithiazolone, phthalimide, quinolinyl, morpholinyl, benzoxazoyl, diazinyl, triazinyl, quinolinyl, quinoxalinyl, naphthyridinyl, azetidinyl, pyrrolidinyl, hydantoinyl, oxathiolanyl, dioxolanyl, imidazolidinyl, and azabicyclo[2.2.1]heptyl.

The term “heteroaryl” refers to an aromatic het, examples of which include, but are not limited to, pyridine and thiophene.

The term “substituted alkyl” refers to an alkyl moiety including 1-4 substituents selected from halo, het, cycloalkyl, cycloalkenyl, aryl, —OQ₁₀, —SQ₁₀, —S(O)₂Q₁₀, —S(O)Q₁₀, —OS(O)₂Q₁₀, —C(═NQ₁₀)Q₁₀, —C(═NOH)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —OPO(OH)₂, and —SNQ₁₀Q₁₀. Each of the het, cycloalkyl, cycloalkenyl, and aryl may be optionally substituted with 1-4 substituents independently selected from halo and Q₁₅.

The term “substituted aryl” refers to an aryl moiety having 1-3 substituents selected from —OQ₁₀, —SQ₁₀, —S(O)₂Q₁₀, —S(O)Q₁₀, —OS(O)₂Q₁₀, —C(═NQ₁₀)Q₁₀, —C(═NOH)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl may be optionally substituted with 1-3 substituents selected from halo and Q₁₅.

The term “substituted het” refers to a het moiety including 1-4 substituents selected from —OQ₁₀, —SQ₁₀, —S(O)₂Q₁₀, —S(O)Q₁₀, —OS(O)₂Q₁₀, —C(═NQ₁₀)Q₁₀, —C(═NOH)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The substituted het also may be substituted by one or more ═O or ═S substituents provided that the O or S are bound to ring atoms capable of supporting a double bond between the ring atom and O or S. The het, cycloalkyl, cycloalkenyl, and aryl may be optionally substituted with 1-3 substituents selected from halo and Q₁₅.

The term “substituted alkenyl” refers to a alkenyl moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl may be optionally substituted with 1-3 substituents selected from halo and Q₁₅.

The term “substituted alkoxy” refers to an alkoxy moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl may be optionally substituted with 1-3 substituents selected from halo and Q₁₅.

The term “substituted cycloalkenyl” refers to a cycloalkenyl moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl may be optionally substituted with 1-3 substituents selected from halo and Q₁₅.

The term “substituted amino” refers to an amino moiety in which one or both of the amino hydrogens are replaced with a group selected from —OQ₁₀, —SQ₁₀, —S(O)₂Q₁₀, —S(O)Q₁₀, —OS(O)₂Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, alkyl, substituted alkyl, het, halo, cycloalkyl, cycloalkenyl, and aryl. The het, cycloalkyl, cycloalkenyl, and aryl may be optionally substituted with 1-3 substituents selected from halo and Q₁₅.

Each Q₁₀ is independently selected from —H, alkyl, cycloalkyl, het, cycloalkenyl, and aryl. The het, cycloalkyl, cycloalkenyl, and aryl may be optionally substituted with 1-3 substituents selected from halo and Q₁₃.

Each Q₁₁ is independently selected from —H, halo, alkyl, aryl, cycloalkyl, and het. The alkyl, cycloalkyl, and het may be optionally substituted with 1-3 substituents independently selected from halo, —NO₂, —CN, ═S, ═O, and Q₁₄. The aryl may be optionally substituted with 1-3 substituents independently selected from halo, —NO₂, —CN, and Q₁₄.

Each Q₁₃ is independently selected from Q₁₁, —OQ₁₁, —SQ₁₁, —S(O)₂Q₁₁, —S(O)Q₁₁, —OS(O)₂Q₁₁, —C(═NQ₁₁)Q₁₁, —SC(O)Q₁₁, —NQ₁₁Q₁₁, —C(O)Q₁₁, —C(S)Q₁₁, —C(O)OQ₁₁, —OC(O)Q₁₁, —C(O)NQ₁₁Q₁₁, —C(O)C(Q₁₆)₂OC(O)Q₁₀, —CN, ═O, ═S, —NQ₁₁C(O)Q₁₁, —NQ₁₁C(O)NQ₁₁Q₁₁, —S(O)₂NQ₁₁Q₁₁, —NQ₁₁S(O)₂Q₁₁, —NQ₁₁S(O)Q₁₁, —NQ₁₁SQ₁₁, —NO₂, and —SNQ₁₁Q₁₁, provided that Q₁₃ is not ═O or ═S when Q₁₀ is aryl or a het lacking any atom capable of forming a double bond with O or S.

Each Q₁₄ is —H or a substituent selected from alkyl, cycloalkyl, cycloalkenyl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —NQ₁₆Q₁₆, —C(O)Q₁₆, —C(S)Q₁₆, —C(O)OQ₁₆, —NO₂, —C(O)NQ₁₆Q₁₆, —CN, —NQ₁₆C(O)Q₁₆, —NQ₁₆C(O)NQ₁₆Q₁₆, —S(O)₂NQ₁₆Q₁₆, and —NQ₁₆S(O)₂Q₁₆, The alkyl, cycloalkyl, and cycloalkenyl may be further substituted with ═O or ═S.

Each Q₁₅ is alkyl, cycloalkyl, cycloalkenyl, het, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(O)C(Q₁₆)₂OC(O)Q₁₆, —CN, —NQ₁₆C(O)Q₁₆, —NQ₁₆C(O)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl may be further substituted with ═O or ═S.

Each Q₁₆ is independently selected from —H, alkyl, and cycloalkyl. The alkyl and cycloalkyl may optionally include 1-3 halos.

Specific R₂ substituents include H, F, Cl, Br, —CN, —NH₂, —NO₂, —CH₃.

Specific structures of A include

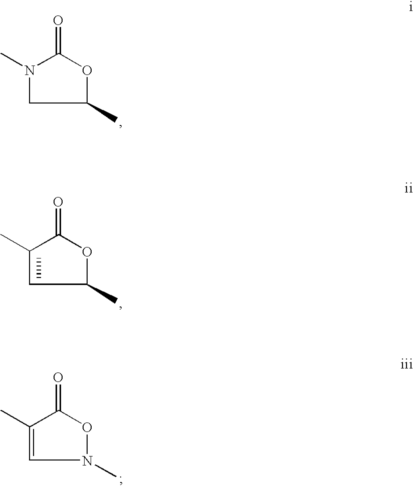

Mammal refers to human or animals.

The compounds of the present invention are generally named according to the IUPAC or CAS nomenclature system. Abbreviations which are well known to one of ordinary skill in the art may be used (e.g. “Ph” for phenyl, “Me” for methyl, “Et” for ethyl, “O” for oxygen atom, “S” for sulfur atom, “N” for nitrogen atom, “h” for hour or hours and “rt” for room temperature).

It is to be understood that the present invention encompasses any racemic, optically-active, polymorphic, tautomeric, or stereoisomeric form, or mixture thereof, of a compound of the invention which possesses the useful properties described herein.

The compounds of the present invention can be converted to their salts, where appropriate, according to conventional methods.

In cases where compounds are sufficiently basic or acidic to form stable nontoxic acid or base salts, use of the compounds as pharmaceutically acceptable salts may be appropriate. Examples of pharmaceutically acceptable salts which are within the scope of the present invention include organic acid addition salts formed with acids which form a physiological acceptable anion and inorganic salts. Examples of pharmaceutically acceptable salts include, but are not limited to, the following acids: acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic and toluenesulfonic. Examples of pharmaceutically acceptable salts include, but are not limited to, the following bases: primary, secondary, and tertiary amines, substituted amines including naturally occurring substituted amines, cyclic amines, arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and the like. The pharmaceutically acceptable salts may be in hydrated form.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of Formula I of this invention contain a chiral center, such as at C-5 of the oxazolidinone ring, and as such there exist two enantiomers or a racemic mixture of both. This invention relates to both the enantiomer that possesses the useful properties described herein, as well as to mixtures containing both of the isomers. In addition, depending on substituents, additional chiral centers and other isomeric forms may be present in any of A, B, or R₁ groups, and this invention embraces all possible stereoisomers and geometric forms in these groups.

The compounds of this invention are useful for treatment of microbial infections in humans and other warm blooded animals.

Dosages and Pharmaceutical Compositions

By the phrase “effective amount” of a compound as provided herein is meant a nontoxic but sufficient amount of one or more compounds of this invention to provide the desired effect. The desired effect may be to prevent, give relief from, or ameliorate microbial infections.

As pointed out below, the exact amount of the compound of this invention required to treat a microbial infection will vary from subject to subject, depending on the species, age, and general condition of the subject, the severity of the disease that is being treated, the particular compound(s) used, and the mode of administration, such as the route and frequency of administration, and the like. Thus, it is not possible to specify an exact “effective amount.” However, an appropriate effective amount may be determined by one of ordinary skill in the art using only routine experimentation.

Pharmaceutical compositions of this invention may be prepared by combining the compounds of this invention with a solid or liquid pharmaceutically acceptable carrier and, optionally, with pharmaceutically acceptable adjuvants and excipients employing standard and conventional techniques. Solid form compositions include powders, tablets, dispersible granules, capsules, cachets and suppositories. A solid carrier can be at least one substance which may also function as a diluent, flavoring agent, solubilizer, lubricant, suspending agent, binder, tablet disintegrating agent, and encapsulating agent. Inert solid carriers include magnesium carbonate, magnesium stearate, talc, sugar, lactose, pectin, dextrin, starch, gelatin, cellulosic materials, low melting wax, cocoa butter, and the like. Liquid form compositions include solutions, suspensions and emulsions. For example, there may be provided solutions of the compounds of this invention dissolved in water and water-propylene glycol systems, optionally containing suitable conventional coloring agents, flavoring agents, stabilizers and thickening agents.

Preferably, the pharmaceutical composition is provided employing conventional techniques in unit dosage form containing effective or appropriate amounts of the active component, that is, the compound according to this invention.

The quantity of active component, that is the compound according to this invention, in the pharmaceutical composition and unit dosage form thereof may be varied or adjusted widely depending upon the particular application, the potency of the particular compound and the desired concentration. Generally, the quantity of active component will range between 0.5% to 90% by weight of the composition.

In therapeutic use for treating, or combating, bacterial infections in warm-blooded animals, the compounds or pharmaceutical compositions thereof will be administered orally, parenterally and/or topically at a dosage to obtain and maintain a concentration, that is, an amount, or blood-level of active component in the animal undergoing treatment which will be antibacterially effective. Generally, such antibacterially effective amount of dosage of active component will be in the range of about 0.1 to about 100, more preferably about 3.0 to about 50 mg/kg of body weight/day. It is to be understood that the dosages may vary depending upon the requirements of the patient, the severity of the bacterial infection being treated, and the particular compound being used. Also, it is to be understood that the initial dosage administered may be increased beyond the above upper level in order to rapidly achieve the desired blood-level or the initial dosage may be smaller than the optimum and the daily dosage may be progressively increased during the course of treatment depending on the particular situation. If desired, the daily dose may also be divided into multiple doses for administration, e.g., 2-4 times per day.

A specific active agent may have more than one recommended dosage range, particularly for different routes of administration. Generally, an effective amount of dosage of compounds of this invention, either administered individually or in combination with other inhibitor compound(s), will be in the range of about 5 to about 2500 mg/day, more specifically about 10 to about 750 mg/day, and most conveniently from 50 to 500 mg per unit dosage form. It is to be understood that the dosages of active component(s) may vary depending upon the requirements of each subject being treated and the severity of the microbial infection.

Initial treatment of a patient suffering from microbial infection can begin with a dosage regimen as indicated above. Treatment is generally continued as necessary over a period of several days to several months until the condition or disorder has been controlled or eliminated. Patients undergoing treatment with a composition of the invention can be routinely monitored by any of the methods well known in the art to determine the effectiveness of therapy. Continuous analysis of data from such monitoring permits modification of the treatment regimen during therapy so that optimally effective amounts of drug are administered at any point in time, and so that the duration of treatment can be determined. In this way, the treatment regimen and dosing schedule can be rationally modified over the course of therapy so that the lowest amount of the compounds of this invention exhibiting satisfactory effectiveness is administered, and so that administration is continued only for so long as is necessary to successfully treat the condition or disorder.

In a combination therapy, the compound(s) of this invention and other inhibitor compound(s) can be administered simultaneously or at separate intervals. When administered simultaneously the compound(s) of this invention and the other inhibitor compound(s) can be incorporated into a single pharmaceutical composition or into separate compositions, e.g., compound(s) of this invention in one composition and the other inhibitor compound(s) in another composition. For instance, the compound(s) of this invention may be administered concurrently or concomitantly with the other inhibitor compound(s). The term “concurrently” means the subject being treated takes one drug within about 5 minutes of taking the other drug. The term “concomitantly” means the subject being treated takes one drug within the same treatment period of taking the other drug. The same treatment period is preferably within twelve hours and up to forty-eight hours.

When separately administered, therapeutically effective amounts of compound(s) of this invention and the other inhibitor compound(s) are administered on a different schedule. One may be administered before the other as long as the time between the two administrations falls within a therapeutically effective interval. A therapeutically effective interval is a period of time beginning when one of either (a) the compound(s) of this invention, or (b) the other inhibitor compound(s) is administered to a mammal and ending at the limit of the beneficial effect in the treatment of microbial infections of the combination of (a) and (b). The methods of administration of the compound(s) of this invention and the other inhibitor compound(s) may vary. Thus, one agent may be administered orally, while the other is administered by injection.

In addition to the compounds of this invention and other antimicrobial agents, the pharmaceutical composition for therapeutic use may also comprise one or more non-toxic, pharmaceutically acceptable carrier materials or excipients. The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, and then tableted or encapsulated for convenient administration. Such capsules or tablets may contain a controlled-release formulation as may be provided in a dispersion of active compound in hydroxypropyl-methyl cellulose, or other methods known to those skilled in the art. For oral administration, the pharmaceutical composition may be in the form of, for example, a tablet, capsule, suspension or liquid. If desired, other active ingredients may be included in the composition.

Routes of Administration

In therapeutic use for treating, or combating, infections in a mammal (i.e., humans and animals) the pharmaceutical composition can be administered orally, parenterally, topically, rectally, or intranasally.

In addition to the oral dosing, noted above, the compositions of the present invention may be administered by any suitable route, in the form of a pharmaceutical composition adapted to such a route, and in a dose effective for the treatment intended. The compositions may, for example, be administered parenterally, e.g., intravascularly, intraperitoneally, subcutaneously, or intramuscularly. For parenteral administration, saline solution, dextrose solution, or water may be used as a suitable carrier. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Generally, the concentration of each of the compounds of this invention in a liquid composition, such as a lotion, will be from about 0.1 wt. % to about 20 wt. %, preferably from about 0.5 wt. % to about 10 wt. %. The solution may contain other ingredients, such as emulsifiers, antioxidants or buffers. The concentration in a semi-solid or solid composition, such as a gel or a powder, will be about 0.1 wt. % to about 5 wt. %, preferably about 0.5 wt. % to about 2.5 wt. %. When topically delivered, the pharmaceutical composition of the present invention being utilized to effect targeted treatment of a specific internal site, each of the compounds of this invention is preferably contained in the composition in an amount of from 0.05-10 wt. %, more preferably 0.5-5 wt. %.

Parenteral administrations include injections to generate a systemic effect or injections directly to the afflicted area. Examples of parenteral administrations are subcutaneous, intravenous, intramuscular, intradermal, intrathecal, intraocular, intravetricular, and general infusion techniques.

Topical administrations include the treatment of infectious areas or organs readily accessibly by local application, such as, for example, eyes, ears including external and middle ear infections, vaginal, open and sutured or closed wounds and skin. It also includes transdermal delivery to generate a systemic effect.

The rectal administration includes the form of suppositories.

The intranasally administration includes nasal aerosol or inhalation applications.

Pharmaceutical compositions including the compounds of this invention may be prepared by methods well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries that facilitate processing of the active compounds into preparations which can be used pharmaceutically. Proper formulation is dependent upon the route of administration chosen.

For oral administration, the compounds of this invention can be formulated by combining the active compounds with pharmaceutically acceptable carriers well known in the art. Such carriers enable the compounds of the invention to be formulated as tablets, pills, lozenges, dragees, capsules, liquids, solutions, emulsions, gels, syrups, slurries, suspensions and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols and other pharmaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identificatin or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono-, di- or triglycerides. Stabilizers may also be added in these formulations.

Liquid form compositions include solutions, suspensions and emulsions. For example, there may be provided solutions of pharmaceutical compositions with the compounds of this invention dissolved in water and water-propylene glycol and water-polyethylene glycol systems, optionally containing suitable conventional coloring agents, flavoring agents, stabilizers and thickening agents.

The compounds of this invention may also be formulated for parenteral administration, e.g., by injections, bolus injection or continuous infusion. Formulations for parenteral administration may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

For injection, the compounds of this invention may be formulated in aqueous solution, preferably in physiologically compatible buffers or physiological saline buffer. Suitable buffering agents include tri-sodium orthophosphate, sodium bicarbonate, sodium citrate, N-methyl-glucamine, L(+)-lysine and L(+)-arginine.

The compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Other parenteral administrations also include aqueous solutions of a water soluble form, such as, without limitation, a salt, of the compounds of this invention. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the compounds of this invention may be in a powder form for constitution with a suitable vehicle (e.g., sterile, pyrogen-free water) before use.

For suppository administration, the pharmaceutical compositions may also be formulated by mixing the compounds of this invention with a suitable non-irritating excipient which is solid at room temperature but liquid at rectal temperature and therefore will melt in the rectum to release the drug. Such materials include cocoa butter, beeswax and other glycerides.

For administration by inhalation, the compounds of this invention can be conveniently delivered through an aerosol spray in the form of solution, dry powder, or cream. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a power base such as lactose or starch.

For ophthalmic and otitis uses, the pharmaceutical compositions may be formulated as micronized suspensions in isotonic, pH adjusted sterile saline, or preferably, as solutions in isotonic, pH adjusted sterile saline, either with or without a preservative, such as benzylalkonium chloride. Alternatively, for ophthalmic uses, the pharmaceutical compositions may be formulated in an ointment, such as petrolatum.

In addition to the formulations described previously, the compounds of this invention may also be formulated as depot preparations. Such long acting formulations may be in the form of implants. The compounds of this invention may be formulated for this route of administration with suitable polymers, hydrophobic materials, or as a sparing soluble derivative such as, without limitation, a sparingly soluble salt.

Additionally, the compounds of this invention may be delivered using a sustained-release system. Various sustained-release materials have been established and are well known by those skilled in the art. Sustained-release capsules may, depending on their chemical nature, release the compounds for 24 hours up to several days. Depending on the chemical nature and the biological stability of the therapeutic reagent, additional strategies for protein stabilization may be employed. In certain embodiments, the compounds of this invention are applied topically. For topical applications, the pharmaceutical composition may be formulated in a suitable ointment containing the compounds of this invention suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion such as suspensions, emulsion, or cream containing the active components suspended or dissolved in one or more pharmaceutically acceptable carriers. Suitable carriers include, but are not limited to, mineral oil, sorbitan monostearate, polysorbate 60, cetyl esters wax, ceteary alcohol, 2-octyldodecanol, benzyl alcohol and water.

In some embodiments, the antibacterial compounds are prodrugs of the compounds of this invention. The expression “prodrug” denotes a derivative of a known direct acting drug, which is transformed into the active drug by an enzymatic or chemical process. Prodrugs of the compounds of this invention are prepared by modifying functional groups present on the compound in such a way that the modifications are cleaved, either in routine manipulation or in vivo, to the parent compound. Prodrugs include, but are not limited to, compounds wherein hydroxy, amine or sulfhydryl groups are bonded to any group that, when administered to the animal, cleaves to form the free hydroxyl, amino or sulfhydryl group, respectively. Representative examples of prodrugs include, but are not limited to, acetate, formate and benzoate derivatives of alcohol and amine functional groups. See Notari, R. E., “Theory and Practice of Prodrug Kinetics,” Methods in Enzymology, 112:309-323 (1985); Bodor, N., “Novel Approaches in Prodrug Design,” Drugs of the Future, 6(3):165-182 (1981); and Bundgaard, H., “Design of Prodrugs: Bioreversible-Derivatives for Various Functional Groups and Chemical Entities,” in Design of Prodrugs (H. Bundgaard, ed.), Elsevier, N.Y. (1985).

The antibacterial agents of this invention have useful activity against a variety of organisms. The in vitro activity of compounds of this invention can be assessed by standard testing procedures such as the determination of minimum inhibitory concentration (MIC) by agar dilution as described in “Approved Standard. Methods for Dilution Antimicrobial Susceptibility Tests for Bacteria That Grow Aerobically”, 3rd. ed., published 1993 by the National Committee for Clinical Laboratory Standards, Villanova, Pa., USA.

Synthetic Methods

Compounds of the present invention may be prepared by the known methodologies illustratived in Chart I and Chart II. Those skilled in the art will promptly recognize appropriate variations from the procedures both as to reaction conditions and techniques. Without further elaboration, it is believed that one skilled in the art can, using the illustrations, practice the present invention to its fullest extent.

As shown in Chart I, treatment of an amide with tosyl chloride in pyridine produces the cyano analogue. The amide may be produced by methods known in the art. CHART II illustrates a method for converting acetamide containing compounds to the cyano analogues. The carboxamide may be hydrolyzed to the amine such as with HCL in methanol followed by ammonia in methanol. The amine may be converted to the nitrile via treatment of NBS and DBU.

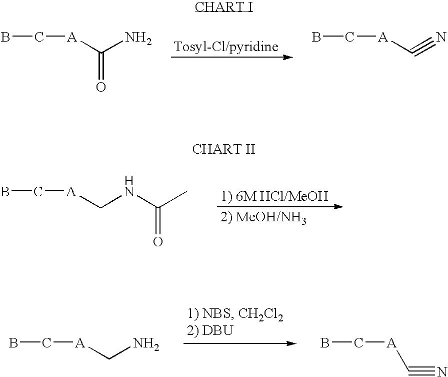

Using the methods described above, the following compounds were made.

### EXAMPLE 1

### (5R)-3-[4-(1,1-dioxidotetrahydro-2H-thiopyran-4-yl)-3-fluorophenyl]-2-oxo-1,3-oxazolidine-5-carbonitrile

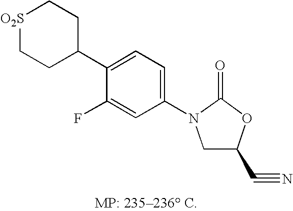

### EXAMPLE 2

### (5R)-3-(3-fluoro-4-morpholin-4-ylphenyl)-2-oxo-1,3-oxazolidine-5-carbonitrile

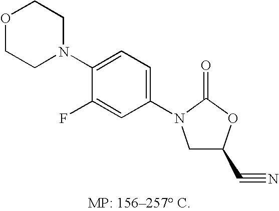

### EXAMPLE 3

### (5R)-3-[4-(1,1-dioxidothiomorpholin-4-yl)-3,5-difluorophenyl]-2-oxo-1,3-oxazolidine-5-carbonitrile

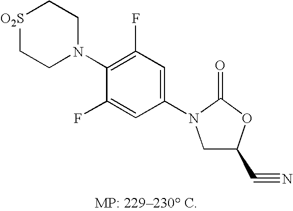

### EXAMPLE 4

### (5R)-3-[3-fluoro-4-(5-methyl-1,3,4-thiadiazol-2-yl)phenyl]-2-oxo-1,3-oxazolidine-5-carbonitrile

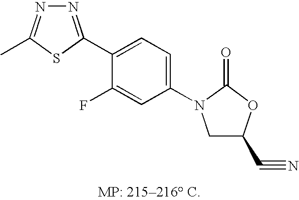

### EXAMPLE 5

### (5R)-3-(3-fluoro-4-pyridin-4-ylphenyl)-2-oxo-1,3-oxazolidine-5-carbonitrile

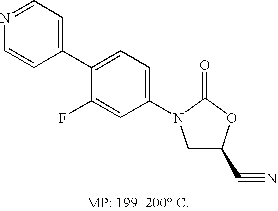

Suitable starting compounds/intermediates useful in preparing compounds of the presention invention and additional synthetic methods to assist in producing the compounds may be found, for example, in the following publications, each of which is hereby incorporated by reference.

U.S. patent application Ser. No. 10/373,286, published on Mar. 4, 2004 (Publication No. US-2004-0044052-A1); U.S. patent application Ser. No. 10/646,440.

U.S. Pat. Nos. 5,225,565; 5,182,403; 5,164,510; 5,247,090; 5,231,188; 5,565,571; 5,547,950; 5,529,998; 5,627,181; 5,843,967; 5,861,413; 5,827,857; 5,869,659; 5,952,324; 5,968,962; 5,688,792; 6,069,160; 6,239,152; 5,792,765; 4,705,799; 5,043,443; 5,652,238; 5,827,857; 5,529,998; 5,684,023; 5,627,181; 5,698,574; 6,166,056; 6,194,441; 6,110,936; 6,069,145; 6,271,383; 5,981,528; 6,051,716; 6,043,266; 6,313,307; 5,614,535; 6,239,283; 5,990,136; and 5,523,403.

Suitable starting compounds/intermediates useful in preparing compounds of the present invention may also be prepared by the procedures described in PCT Application and publications PCT/US93/04850, WO94/01 110; PCT/US94/08904, WO95/07271; PCT/US95/02972, WO95/25106; PCT/US95/10992, WO96/13502; PCT/US96/05202, WO96/35691; PCT/US96/12766; PCT/US96/13726; PCT/US96/14135; PCT/US96/17120; PCT/US96/19149; PCT/US97/01970; PCT/US95/12751, WO96/15130, PCT/US96/00718, WO96/23788, WO98/54161, WO99/29688, WO99/03846, WO99/37641, WO99/37652, WO99/40094, WO97/30995, WO97/09328, WO01/81350, WO01/40236, WO00/21960 WO01/04022, WO00/73301, WO01/46185, WO99/64417, WO02/59155, WO02/096916 and WO95/07271.

In some embodiments, the antibacterial compounds are prodrugs of the compounds of formulae I, II, and III. The expression “prodrug” denotes a derivative of a known direct acting drug, which is transformed into the active drug by an enzymatic or chemical process. Prodrugs of the compounds of formulea I, II, and III are prepared by modifying functional groups present on the compound in such a way that the modifications are cleaved, either in routine manipulation or in vivo, to the parent compound. Prodrugs include, but are not limited to, compounds of structure I, II, and III wherein hydroxy, amine or sulfhydryl groups are bonded to any group that, when administered to the animal, cleaves to form the free hydroxyl, amino or sulfhydryl group, respectively. Representative examples of prodrugs include, but are not limited to, acetate, formate and benzoate derivatives of alcohol and amine functional groups. See Notari, R. E., “Theory and Practice of Prodrug Kinetics,” Methods in Enzymology, 112:309-323 (1985); Bodor, N., “Novel Approaches in Prodrug Design,” Drugs of the Future, 6(3):165-182 (1981); and Bundgaard, H., “Design of Prodrugs: Bioreversible-Derivatives for Various Functional Groups and Chemical Entities,” in Design of Prodrugs (H. Bundgaard, ed.), Elsevier, N.Y. (1985).

MIC Test Method

The in vitro MICs of test compounds are determined by a standard agar dilution method. A stock drug solution of each analog is prepared in the preferred solvent, usually DMSO:H₂O (1:3). Serial 2-fold dilutions of each sample are made using 1.0 ml aliquots of sterile distilled water. To each 1.0 ml aliquot of drug is added 9 ml of molten Mueller Hinton agar medium. The drug-supplemented agar is mixed, poured into 15×100 mm petri dishes, and allowed to solidify and dry prior to inoculation.

Vials of each of the test organisms are maintained frozen in the vapor phase of a liquid nitrogen freezer. Test cultures are grown overnight at 35° C. on the medium appropriate for the organism. Colonies are harvested with a sterile swab, and cell suspensions are prepared in Trypticase Soy broth (TSB) to equal the turbidity of a 0.5 McFarland standard. A 1:20 dilution of each suspension is made in TSB. The plates containing the drug supplemented agar are inoculated with a 0.001 ml drop of the cell suspension using a Steers replicator, yielding approximately 10⁴ to 10⁵ cells per spot. The plates are incubated overnight at 35° C.

Following incubation the Minimum Inhibitory Concentration (MIC μg/ml), the lowest concentration of drug that inhibits visible growth of the organism, is read and recorded. The data is shown in Table I.

TABLE 1
MIC (μg/ml) of Compounds of Formula I for Staphylococcus aureus
(SAUR 9213)

| Example No. | SAUR 9213 MIC (μg/ml) |
|---|---|
| 1 | 1 |
| 2 | 8 |
| 3 | 2 |
| 4 | 4 |
| 5 | 0.5 |

1. A compound of formula I 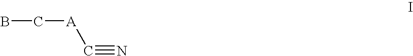
or a pharmaceutically acceptable salt thereof wherein:
A is a structure i, ii, or iii 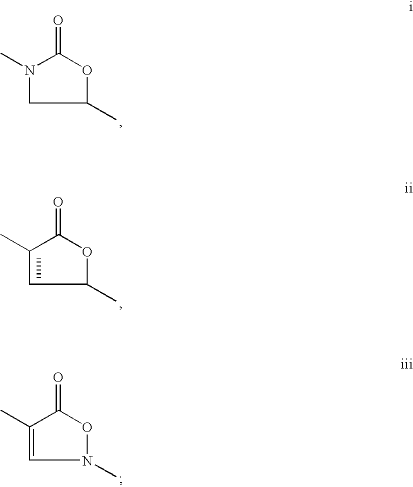
C is aryl or heteroaryl, wherein each of the aryl and heteroaryl are optionally substituted with 1-3 of R₂;
B is selected from cycloalkyl, substituted cycloalkyl, cycloalkenyl, substituted cycloalkenyl, aryl, substituted aryl, het and substituted het, or B and one R₂, if present, together, with the phenyl carbon atoms to which B and the one R₂ are bonded, form a het, the het optionally being a substituted het; and
Each R₂ is independently selected from H, alkyl, amino, NO₂, —CN, halo, and substituted alkyl.

2. A compound of formula II 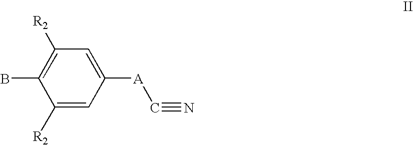
or a pharmaceutically acceptable salt thereof wherein:
A is a structure i, ii, or iii 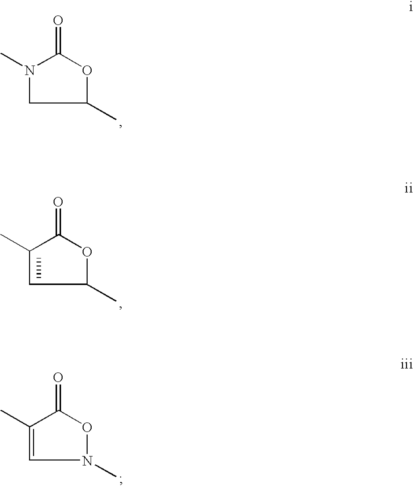
B is selected from cycloalkyl, substituted cycloalkyl, cycloalkenyl, substituted cycloalkenyl, aryl, substituted aryl, het, and substituted het, or B and one R₂ together, with the phenyl carbon atoms to which B and the one R₂ are bonded, form a het, the het optionally being a substituted het; and
Each R₂ is independently selected from H, alkyl, amino, NO₂, —CN, halo, and substituted alkyl.

3. The compound of claim 2, wherein each R₂ is independently selected from H, F, Cl, Br, CN, NH₂, NO₂, CF₃, and CH₃.

4. The compound of claim 2, wherein the structure of A is

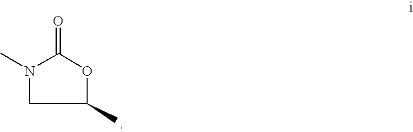

5. The compound of claim 4, wherein B is morpholinyl, piperazinyl, pyridyl, thiomorpholinyl, 3,6-dihydro-2H-thiopyranyl, tetrahydro-2H-thiopyranyl, 3,6-dihydro-2H-pyranyl, tetrahydro-2H-pyranyl, azetidinyl, 5,6-dihydro-4H-[1,3,4]thiadiazinyl, 2,5-dihydro-1H-pyrrolyl, 3,4-dihydro-1(2H)-pyridinyl, tetrahydropyridyl, 5,7-dihydro-6H-pyrrolo[3,4-b]pyridinyl, 2,3-dihydro-4H-1,4-thiazinyl, each of the morpholinyl, piperazinyl, pyridyl, thiomorpholinyl, 3,6-dihydro-2H-thiopyranyl, tetrahydro-2H-thiopyranyl, 3,6-dihydro-2H-pyranyl, tetrahydro-2H-pyranyl, azetidinyl, 5,6-dihydro-4H-[1,3,4]thiadiazinyl, 2,5-dihydro-1H-pyrrolyl, 3,4-dihydro-1(2H)-pyridinyl, tetrahydropyridyl, 5,7-dihydro-6H-pyrrolo[3,4-b]pyridinyl, 2,3-dihydro-4H-1,4-thiazinyl being optionally substituted with 1-4 groups selected from ═O, alkyl, substituted alkyl, amino, substituted amino, —OH, ═NOH, ═NC₁₋₄ alkyl, and halo.

6. The compound of claim 4, wherein B is selected from

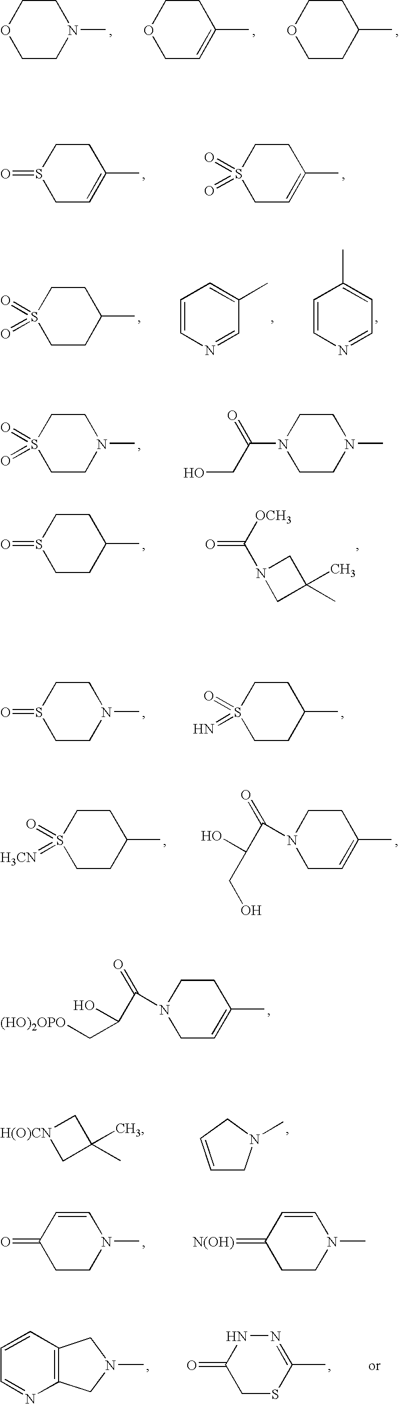

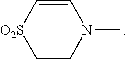

7. The compound of claim 4, wherein each R₂ is independently H or F.

8. A compound of formula III 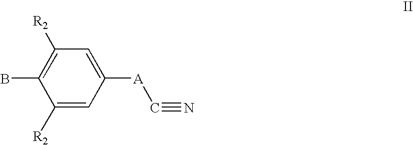
or a pharmaceutically acceptable salt thereof wherein:
A is a structure i, ii, or iii 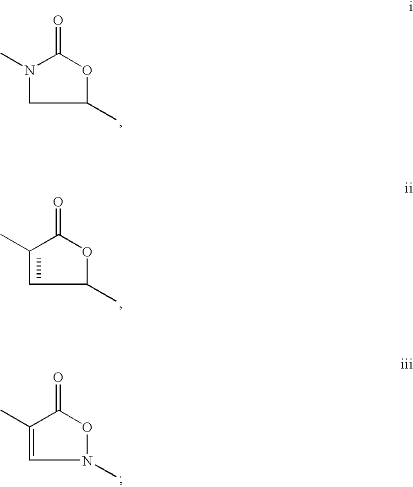
B is 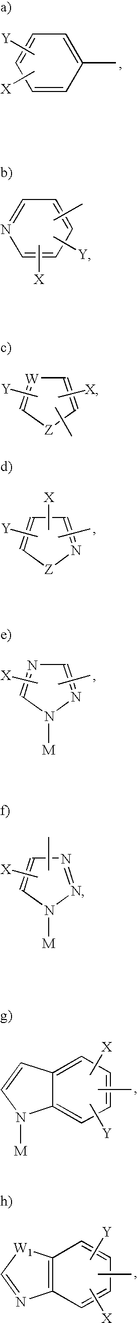 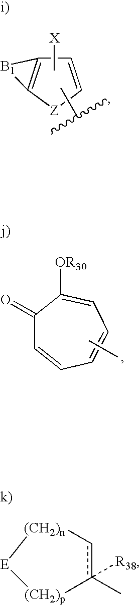
l) a diazinyl group optionally substituted with X and Y,
m) a triazinyl group optionally substituted with X and Y,
n) a quinolinyl group optionally substituted with X and Y,
o) a quinoxalinyl group optionally substituted with X and Y,
p) a naphthyridinyl group optionally substituted with X and Y, 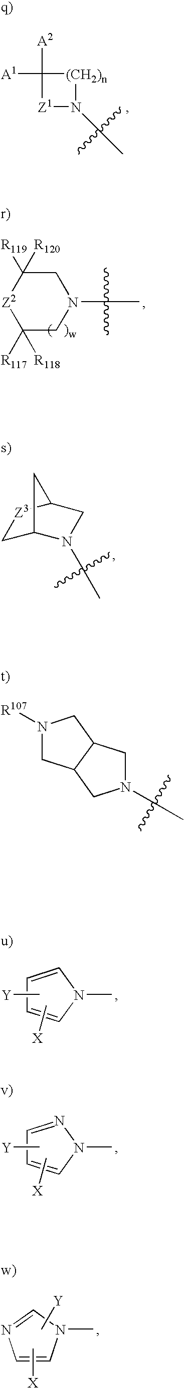 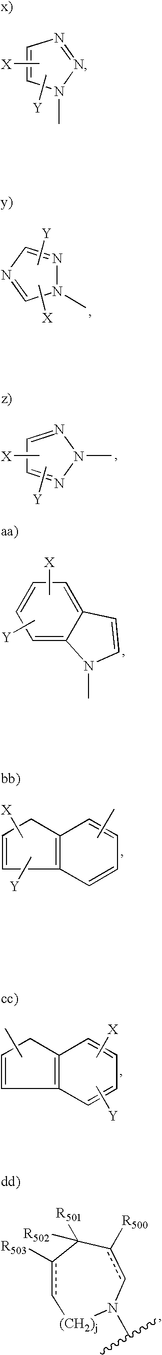 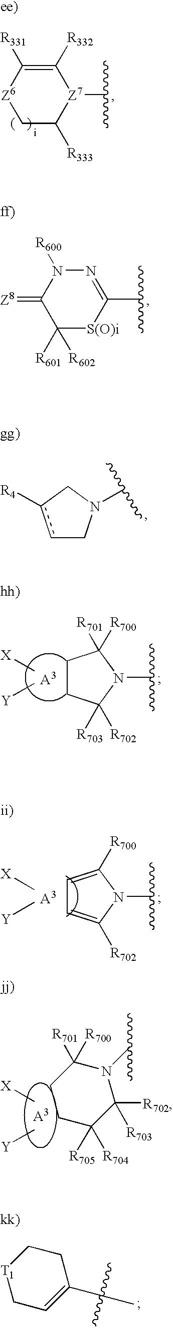 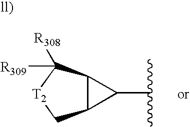
B and one R₂ are taken together to form 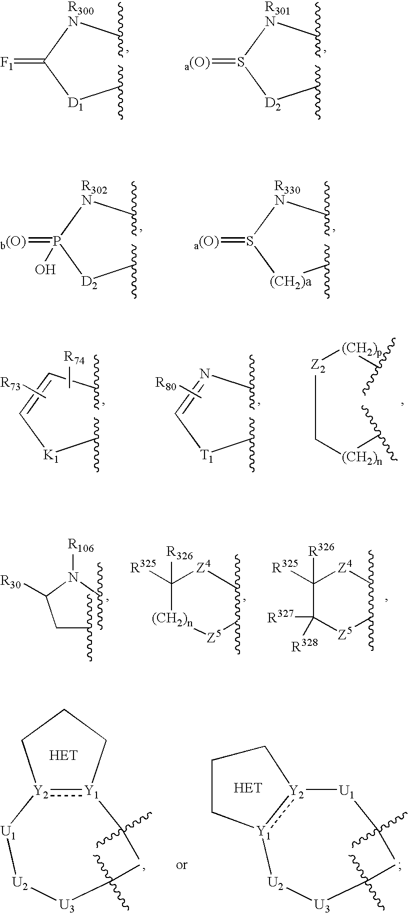
A¹ is
a) H—, or
b) CH₃;
A² is
a) H—,
b) HO—,
c) CH₃—,
d) CH₃O—,
e) R¹⁰²O—CH₂—C(O)—NH—
f) R¹⁰³O—C(O)—NH—,
g) (C₁-C₂)alkyl-O—C(O)—,
h) HO—CH₂—,
i) CH₃O—NH—,
j) (C₁-C₃)alkyl-O₂C—
k) CH₃—C(O)—,
l) CH₃—C(O)—CH₂—, 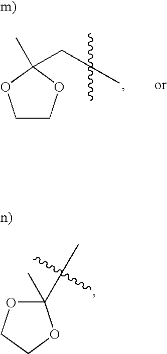
A¹ and A² taken together are: 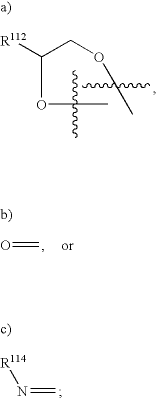
A³ represents any 5-10 membered aryl ring or aromatic het, the het having 1-4 heteroatoms selected from O, S, or N;
B₁ is
a) —N═C(H)—C(H)═C(H)—, or
b) —C(H)═N—C(H)═C(H)—;
D₁ is
a) O,
b) S(O)ᵢ, or
c) —N(R₃₀₄)—;
D₂ is
a) O, or
b) —N(R₃₀₄)—;
E is
a) NR₃₉,
b) —S(═O)ᵢ,
c) O, or
d) —S(═O)(═NR₃₁₅);
F₁ is
a) O,
b) S,
c) NH,
d) N—OH,
e) N—O—C₁₋₄ alkyl,
f) N—OC(O)—C₁₋₄ alkyl, or
g) N—C₁₋₄alkyl;
K₁ is
a) O,
b) S, or
c) —NR₃₀₅—;
M is
a) H,
b) C₁₋₈ alkyl,
c) C₃₋₈ cycloalkyl,
d) —(CH₂)ₘOR₁₃, or
e) —(CH₂)ₕ—NR₂₁R₂₂;
T₁ is
a) —O—,
b) —NR₃₀₆—
c) —S(O)ᵢ—,
d) —C(R₂)—, or
e) —C(O)—;
T₂ is
a) —O—,
b) —NR₃₀₇—,
c) —S(O)ᵢ—,
d) —C(O)—, or
e) —C(R₂)₂—;
Each U₁, U₂, and U₃ is independently selected from
a) —C(R₁)₂—,
b) —NR₃₉—,
c) —O—, or
d) —S(O)ᵢ—;
V is
a) O,
b) CH₂, or
c) NR₈₇;
W is
a) CH, or
b) N;
W₁ is
a) —NH—,
b) O, or
c) S;
W₂ is O or S;
X is
a) H,
b) —CN,
c) —OR₂₇,
d) halo,
e) —NO₂,
f) tetrazolyl optionally substituted with C₁₋₄alkyl,
g) —SH,
h) —S(═O)ᵢR₄,
i) —SC(═O)R₇,
j) —C(═O)R₂₅,
k) —C(═O)NR₂₇R₂₈,
l) —C(═NR₂₉)R₂₅,
m) —C(R₂₅)(R₂₈)—OR₁₃,
n) —C(R₂₅)(R₂₈)—OC(═O)R₁₃,
o) —C(R₂₈)(OR₁₃)—(CH₂)ₕ—NR₂₇R₂₈,
p) —NR₂₇R₂₈,
q) —N(R₂₇)C(═O)R₇,
r) —N(R₂₇)—S(═O)ᵢR₇,
s) —C(OR₁₄)(OR₁₅)R₂₈,
t) —C(R₂₅)(R₁₆)—NR₂₇R₂₆,
u) —C₁₋₈ alkyl substituted with one or more halos, OH, ═O other than at alpha position, —S(═O)ᵢR₁₇, —NR₂₇R₂₈, C₂₋₅ alkenyl, C₂₋₅ alkynyl, or C₃₋₈ cycloalkyl, or
v) -Het optionally substituted with R₂, ═O or ═S;
Y is
a) H,
b) F,
c) Cl,
d) Br,
e) C₁₋₃ alkyl, or
f) NO₂;
Each Y₁ and Y₂ is independently
a) CH, or
b) N provided that . . . is absent, or
c) C when . . . is present;
Z is
a) O,
b) S, or
c) NM;
Z₁ is
a) —CH₂—,
b) —CH(R¹⁰⁴)—CH₂—,
c) —C(O)—, or
d) —CH₂CH₂CH₂—;
Z² is
a) —S(O)i-,
b) —O—,
c) —N(R¹⁰⁷)—, or
d) —S(═O)(═NR³¹⁵)—;
Z³ is
a) —S(O)i-, or
b) —O—,
Z⁴ is
a) —S(═O)i-, or
b) —NR³⁰³—;
Z⁴ is
a) —O—,
b) —NH—,
c) —CH₂—,
d) —C(halo)₂-, or
e) —S(═O)ᵢ—;
Z⁶ is
a) S(═O)ᵢ,
b) S(═NR³¹⁵), or
c) S(═NR³¹⁵)(═O);
Z⁷ is
a) N,
b) CR¹¹⁰,
c) CR¹¹⁵, or
d) CR¹¹⁶
Z⁸ is
a) O, or
b) S;
R₁ is
a) H,
b) —OH,
c) C₁₋₆ alkyl optionally substituted with one or more halos, —OH, —CN, aryl, het, alkoxy, substituted aryl or substituted het,
d) C₁₋₆ alkoxy optionally substituted with one or more halos, —OH, —CN, aryl, het, substituted aryl or substituted het,
e) C₂₋₆ alkenyl optionally substituted with aryl, het, substituted aryl or substituted het,
f) —NH₂, or
g) C₃₋₅ cycloalkyl;
R₂ is
a) H,
b) C₁₋₂ alkyl optionally substituted with one or more halos,
c) —NH₂,
d) —NO₂,
e) —CN, or
f) halo;
R₄ is
a) H
b) C₁₋₄ alkyl optionally substituted with one or more halos, OH, CN, NR₁₀R₁₁, or —CO₂R₁₃,
c) C₂₋₄ alkenyl,
d) —NR₁₆R₁₈,
e) —NHC(═O)R₇,
f) —NR₂₀C(═O)R₇,
g) —N(R₁₇)₂,
h) —NR₁₆R₁₇, or
i) —NR₁₇R₂₀;
R₅ and R₆ at each occurrence are the same or different and are
a) C₁₋₂ alkyl, or
b) R₅ and R₆ taken together are —(CH₂)ₖ—;
R₇ is
a) C₁₋₄ alkyl optionally substituted with one or more halos;
R₁₀ and R₁₁ at each occurrence are the same or different and are
a) H,
b) C₁₋₄ alkyl, or
c) C₃₋₈ cycloalkyl;
R₁₃ is
a) H, or
b) C₁₋₄ alkyl;
R₁₄ and R₁₅ at each occurrence are the same or different and are
a) C₁₋₄ alkyl, or
b) R₁₄ and R₁₅ taken together are —(CH₂)₁—;
R₁₆ is
a) H,
b) C₁₋₄ alkyl, or
c) C₃₋₈ cycloalkyl;
R₁₇ is
a) C₁₋₄ alkyl, or
b) C₃₋₈ cycloalkyl;
R₁₈ is
a) H,
b) C₁₋₄ alkyl,
c) C₂₋₄ alkenyl,
d) C₃₋₄ cycloalkyl,
e) —OR₁₃ or
f) —NR₂₁R₂₂;
R₂₀ is a physiologically acceptable cation, such as sodium, potassium, lithium, calcium or magnesium;
R₂₁ and R₂₂ at each occurrence are the same or different and are
a) H,
b) C₁₋₄ alkyl, or
c) R₂₁ and R₂₂ taken together are —(CH₂)ₘ—;
R₂₅ is
a) H,
b) C₁₋₈ alkyl optionally substituted with one or more halos, C₃₋₈ cycloalkyl, C₁₋₄ alkyl substituted with one or more of —S(═O)ᵢR₁₇, —OR₁₃, or OC(═O)R₁₃, NR₂₇R₂₈, or
c) C₂₋₅ alkenyl optionally substituted with —C(O)H, or CO₂R₁₃;
R₂₆ is
a) R₂₈, or
b) —NR₂₇N₂₈;
R₂₇ and R₂₈ at each occurrence are the same or different and are
a) H,
b) C₁₋₈ alkyl,
c) C₃₋₈ cycloalkyl,
d) —(CH₂)ₘOR₁₃,
e) —(CH₂)ₕ—NR₂₁R₂₂, or
f) R₂₇ and R₂₈ taken together are —(CH₂)₂O(CH₂)₂—, —(CH₂)ₕCH(COR₇)—, or —(CH₂)₂N(CH₂)₂(R₇);
R₂₉ is
a) —NR₂₇R₂₈,
b) —OR₂₇, or
c) —NHC(═O)R₂₈;
R₃₀ is
a) H, or
b) C₁₋₄ alkyl optionally substituted with one or more halos, OH, C₁₋₄ alkoxy, CN, SH, NH₂, —OR₃₁, —NHR₃₁, —N(R₃₁)₂, or —S(O)iR₃₁;
R₃₁ is
a) C₁₋₄ alkyl,
b) —C(O)C₁₋₄ alkyl,
c) —C(O)OC₁₋₄ alkyl,
d) —C(O)NH₂,
e) —C(O)NHC₁₋₄ alkyl, or
f) —SO₂C₁₋₄ alkyl;
R₃₈ is
a) H,
b) C₁₋₆ alkyl,
c) —(CH₂)_{q}-aryl, or
d) halo;
R₃₉ is
a) H,
b) C₁₋₆ alkyl optionally substituted with one or more OH, halo, or —CN,
c) —(CH₂)_{q}-aryl,
d) —CO₂R₄₀,
e) —COR₄₁,
f) —C(═O)—(CH₂)_{q}—C(═O)R₄₀,
g) —S(═O)₂—C₁₋₆alkyl,
h) —S(═O)₂—(CH₂)_{q}-aryl, or
i) —(C═O)ⱼ-Het;
R₄₀ is
a) H,
b) C₁₋₆ alkyl optionally substituted with one or more OH, halo, or —CN,
c) —(CH₂)_{q}-aryl, or
d) —(CH₂)_{q}—OR₄₂;
R₄₁ is
a) C₁₋₆ alkyl optionally substituted with one or more OH, halo, —OP(O)(OH)₂, —OP(OH)₂, or —CN,
b) —(CH₂)_{q}-aryl, or
c) —(CH₂)_{q}—OR₄₂;
R₄₂ is
a) H,
b) C₁₋₆ alkyl,
c) —(CH₂)_{q}-aryl, or
d) —C(═O)—C₁₋₆ alkyl;
R₄₉ and R₅₀ at each occurrence are the same or different and are
a) H,
b) C₁₋₄ alkyl,
c) C₅₋₆ cycloalkyl, or
d) R₄₉ and R₅₀ taken together with the nitrogen atom is a 5-, 6-membered saturated heterocyclic moiety which optionally has a further hetero atom selected from the group consisting of S, N, and O, and can in turn be optionally substituted with, including on the further nitrogen atom, C₁₋₃ alkyl, or C₁₋₃ acyl;
R₅₁ is
a) carboxyl,
b) halo,
c) —CN,
d) mercapto,
e) formyl,
f) CF₃,
g) —NO₂,
h) C₁₋₆ alkoxy,
i) C₁₋₆ alkoxycarbonyl,
j) C₁₋₆ alkythio,
k) C₁₋₆ acyl,
l) C₁₋₆ alkyl optionally substituted with OH, C₁₋₅ alkoxy, C₁₋₅ acyl, or —NR₄₉R₅₀,
m) phenyl,
n) —C(═O)NR₅₂R₅₃,
o) —NR₄₉R₅₀,
P) —N(R₅₂)(—SO₂R₅₄),
q) —SO₂—NR₅₂R₅₃, or
r) —S(═O)ᵢR₅₄;
R₅₂ and R₅₃ at each occurrence are the same or different and are
a) H,
b) C₁₋₆ alkyl, or
c) phenyl;
R₅₄ is
a) C₁₋₄ alkyl, or
b) phenyl optionally substituted with C₁₋₄ alkyl;
R₇₃ and R₇₄ at each occurrence are the same or different and are
a) H,
b) carboxyl,
c) halo,
d) —CN,
e) mercapto,
f) formyl,
g) CF₃,
h) —NO₂,
i) C₁₋₆ alkoxy,
j) C₁₋₆ alkoxycarbonyl,
k) C₁₋₆ alkythio,
l) C₁₋₆ acyl,
m) —NR₇₈R₇₉,
n) C₁₋₆ alkyl optionally substituted with OH, C₁₋₅ alkoxy, C₁₋₅ acyl, —NR₇₈R₇₉, —N(phenyl)(CH₂—CH₂—OH), —O—CH(CH₃)(OCH₂CH₃), or —O-phenyl-[para-NHC(═O)CH₃],
o) C₂₋₈ alkenylphenyl optionally substituted with R₅₁,
p) phenyl optionally substituted with R₅₁, or
q) a 5-, or 6-membered saturated or unsaturated heterocyclic moiety having one to three atoms selected from the group consisting of S, N, and O, optionally substituted with R₅₁;
R₇₈ and R₇₉ at each occurrence are the same or different and are
a) H,
b) C₁₋₄ alkyl,
c) phenyl, or
d) R₇₈ and R₇₉ taken together with the nitrogen atom is a 5-, 6-membered saturated heterocyclic moiety which optionally has a further hetero atom selected from the group consisting of S, N, and O, and can in turn be optionally substituted with, including on the further nitrogen atom, C₁₋₃ alkyl, or C₁₋₃ acyl;
R₈₀ is
a) H,
b) formyl,
c) carboxyl,
d) C₁₋₆ alkoxycarbonyl,
e) C₁₋₈ alkyl,
f) C₂₋₈ alkenyl,
wherein the substituents (e) and (f) can be optionally substituted with OH, halo, C₁₋₆ alkoxy, C₁₋₆ acyl, C₁₋₆ alkylthio or C₁₋₆ alkoxycarbonyl, or phenyl optionally substituted with halo,
g) an aromatic moiety having 6 to 10 carbon atoms optionally substituted with carboxyl, halo, —CN, formyl, CF₃, —NO₂, C₁₋₆ alkyl, C₁₋₆ alkoxy, C₁₋₆ acyl, C₁₋₆ alkylthio, or C₁₋₆ alkoxycarbonyl;
h) —NR₈₁R₈₂,
i) —OR₉₀,
j) —S(═O)ᵢ—R₉₁, or
k) —SO₂—N(R₉₂)(R₉₃);
R₈₁ and R₈₂ at each occurrence are the same or different and are
a) H,
b) C₃₋₆ cycloalkyl,
c) phenyl,
d) C₁₋₆ acyl,
e) C₁₋₈ alkyl optionally substituted with OH, C₁₋₆ alkoxy which can be substituted with OH, a 5-, or 6-membered aromatic heterocyclic moiety having one to three atoms selected from the group consisting of S, N, and O, phenyl optionally substituted with OH, CF₃, halo, —NO₂, C₁₋₄ alkoxy, —NR₈₃R₈₄, or 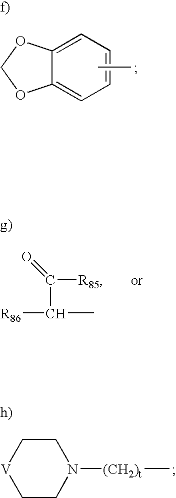
R₈₃ and R₈₄ at each occurrence are the same or different and are
a) H, or
b) C₁₋₄ alkyl;
R₈₅ is
a) OH,
b) C₁₋₄ alkoxy, or
c) —NR₈₈R₈₉;
R₈₆ is
a) H, or
b) C₁₋₇ alkyl optionally substituted with indolyl, OH, mercaptyl, imidazoly, methylthio, amino, phenyl optionally substituted with OH, —C(═O)—NH₂, —CO₂H, or —C(═NH)—NH₂;
R₈₇ is
a) H,
b) phenyl, or
c) C₁₋₆ alkyl optionally substituted by OH;
R₈₈ and R₈₉ at each occurrence are the same or different and are
a) H,
b) C₁₋₅ alkyl
c) C₃₋₆ cycloalkyl, or
d) phenyl;
R₉₀ is
a) C₁₋₈ alkyl optionally substituted with C₁₋₆ alkoxy or C₁₋₆ hydroxy, C₃₋₆ cycloalkyl, a 6-membered aromatic optionally benzo-fused heterocyclic moiety having one to three nitrogen atoms, which can in turn be substituted with one or two —NO₂, CF₃, halo, —CN, OH, C₁₋₅ alkyl, C₁₋₅ alkoxy, or C₁₋₅ acyl; 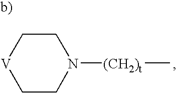
c) phenyl, or
d) pyridyl;
R₉₁ is
a) C₁₋₁₆ alkyl,
b) C₂₋₁₆ alkenyl,
wherein the substituents (a) and (b) can be optionally substituted with C₁₋₆ alkoxycarbonyl, or a 5-, 6-, 7-membered aromatic heterocyclic moiety having one to three atoms selected from the group consisting of S, N, and O,
c) an aryl having 6 to 10 carbon atoms, or
d) a 5-, 6-, 7-membered aromatic heterocyclic moiety having one to three atoms selected from the group consisting of S, N, and O, wherein the substituents (c) and (d) can be optionally substituted with carboxyl, halo, —CN, formyl, CF₃, —NO₂, C₁₋₆ alkyl, C₁₋₆ alkoxy, C₁₋₆ acyl, C₁₋₆ alkylthio, or C₁₋₆ alkoxycarbonyl;
R₉₂ and R₉₃ at each occurrence are the same or different and are
a) H,
b) phenyl,
c) C₁₋₆ alkyl, or
d) benzyl;
R¹⁰² is
a) H—,
b) CH₃—,
c) phenyl-CH₂—, or
d) CH₃C(O)—;
R¹⁰³ is
a) (C₁-C₃)alkyl-, or
b) phenyl-;
R¹⁰⁴ is
a) H—, or
b) HO—;
R¹⁰⁶ is
a) CH₃—C(O)—,
b) H—C(O)—,
c) Cl₂CH—C(O)—,
d) HOCH₂—C(O)—,
e) CH₃SO₂—, 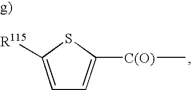
h) F₂CHC(O)—, 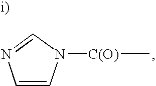
j) H₃C—C(O)—O—CH₂—C(O)—,
k) H—C(O)—O—CH₂—C(O)—, 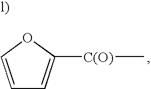
m HC≡C—CH₂O—CH₂—C(O)—,
n) phenyl-CH₂—O—CH₂—C(O)—,
o) C₁₋₄alkyl-NH—C(S)—, or
p) C₁₋₄alkyl optionally substituted with one or more halo, CN, NO₂, OH, SH, or NH₂;
R¹⁰⁷ is
a) R¹⁰²O—C(R¹¹⁰)(R¹¹¹)—C(O)—,
b) R¹⁰³O—C(O)—,
c) R¹⁰⁸—C(O)—, 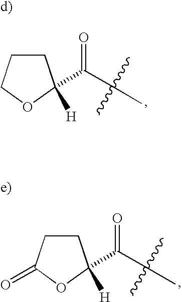
f) H₃C—C(O)—(CH₂)₂—C(O)—,
g) R¹⁰⁹—SO₂—, 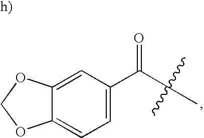
i) HO—CH₂—C(O)—,
i) R¹¹⁶—(CH₂)₂—,
k) R¹¹³—C(O)—O—CH₂—C(O)—,
l) (CH₃)₂N—CH₂—C(O)—NH—,
m) NC'CH₂—,
n) F₂—CH—CH₂—, or
o) R¹⁵⁰R¹⁵¹NSO₂
R¹⁰⁸ is
a) H—,
b) (C₁-C₄)alkyl,
c) aryl-(CH₂)ₙ,
d) ClH₂C—,
e) Cl₂HC—,
f) FH₂C—,
g) F₂HC—,
h) (C₃-C₆)cycloalkyl, or
i) CNCH₂—.
R¹⁰⁹ is
a) C₁-C₄alkyl,
b) —CH₂Cl
c) —CH₂CH═CH₂,
d) aryl, or
e) —CH₂CN;
R¹¹⁰ and R¹¹¹ are independently
a) H—,
b) CH₃—; or
R¹¹² is
a) H—,
b) CH₃O—CH₂O—CH₂—, or
c) HOCH₂—;
R¹¹³ is
a) CH₃—,
b) HOCH₂—,
c) (CH₃)₂N-phenyl, or
d) (CH₃)₂N—CH₂—;
R¹¹⁴ is
a) HO—,
b) CH₃O—,
c) H₂N—,
d) CH₃O—C(O)—O—,
e) CH₃—C(O)—O—CH₂—C(O)—O—,
f) phenyl-CH₂—O—CH₂—C(O)—O—,
g) HO—(CH₂)₂—O—,
h) CH₃O—CH₂—O—(CH₂)₂—O—, or
i) CH₃O—CH₂—O—;
R¹¹⁵ is
a) H—, or
b) Cl—;
R¹¹⁶ is
a) HO—
b) CH₃—, or
c) F;
Each of R₁₁₇, R,₁₁₈, R₁₁₉, and R₁₂₀ is independently selected from
a) H,
b) C₁-C₆alkyl,
c) substituted alkyl,
d) halo, or
e) R₁₁₇ and R₁₁₈ or R₁₁₉ and R₁₂₀ together are ═O;
R¹⁵⁰ and R¹⁵¹ are each independently
a) H,
b) C₁-C₄alkyl, or
c) R¹⁵⁰ and R¹⁵¹ taken together with the nitrogen atom, to which R¹⁵⁰ and R¹⁵¹ are attached, form a monocyclic heterocyclic ring having from 3 to 6 carbon atoms;
Each R₃₀₀, R₃₀₁, R₃₀₂, R₃₀₃, R₃₀₄, R₃₀₅, and R₃₀₆ is independently selected from
a) H,
b) C₃₋₆ cycloalkyl optionally substituted with ═O,
c) C₁₋₆ alkoxy,
d) C₁₋₁₀ alkyl optionally substituted with one or more of R₃₁₀,
e) C₂₋₁₀ alkenyl optionally substituted with one or more of R₃₁₀,
f) benzyloxycarbonyl,
g) aryl,
h) het,
i) —C(O)—NR₃₁₁R₃₁₂,
j) —S(O)₂—NR₃₁₁R₃₁₂,
k) —(O)ᵢSR₃₁₁,
l) —C(O)—R₃₁₀,
m) —C(S)—NR₃₁₁R₃₁₂,
n) —C(O)—H, or
o) —C(O)—C₁₋₄alkyl optionally substituted with one or more of R₃₁₀;
Each R₃₀₇ is independently selected from
a) H,
b) C₃₋₆ cycloalkyl optionally substituted with ═O,
c) C₁₋₆ alkoxy,
d) C₁₋₁₀ alkyl optionally substituted with one or more of R₃₁₀,
e) C₂₋₁₀ alkenyl optionally substituted with one or more of R₃₁₀,
f) benzyloxycarbonyl,
g) aryl,
h) het,
i) —C(O)—NR₃₁₁R₃₁₂,
j) —S(O)₂—NR₃₁₁R₃₁₂,
k) —(O)ᵢSR₃₁₁,
l) —C(O)—R₃₁₀,
m) —C(S)—NR₃₁₁R₃₁₂,
n) —C(O)—H,
o) —C(O)—C₁₋₄alkyl optionally substituted with one or more of R₃₁₀, or
p) —C(N—O—C₁₋₄alkyl)-;
R₃₀₈ and R₃₀₉ are H or both R₃₀₈ and R₃₀₉ together form ═O or ═S;
R₃₁₀ is
a) —CN,
b) —N₃,
c) —CF₃,
d) pyridyl,
e) halo,
f) —OH,
g) —O(O)C₁-C₆alkyl,
h) —C₁₋₆ alkyloxycarbonyl,
i) —SH,
j) —NH₂;
Each R₃₁₁ and R₃₁₂ is independently selected from
a) H,
b) C₁₋₄ alkyl,
c) phenyl, or
d) R₃₁₁ and R₃₁₂ together with the N-atom to which they are attached forms a 5- or 6-membered, saturated heterocyclic ring optionally having one or more O, S, or N atoms in the ring, the heterocyclic ring being optionally substituted with C₁₋₃ alkyl;
R₃₁₅ is
a) H,
b) C₁₋₄ alkyl optionally substituted with halo, —OH, C₁₋₈ alkoxy, amino, C₁₋₈ alkylamino, or C₁₋₈dialkylamino,
c) aryl-S(O)₂—,
d) C(═O)C₁₋₄alkyl,
e) C(═O)OC₁₋₄alkyl,
f) C(═O)NHR₃₂₀,
g) C(═S)NHR₃₂₀,
h) —OC(═O)C₁₋₄alkyl,
i) —S(O)ᵢC₁₋₄alkyl,
i) C₁₋₄ alkyl-O—C₁₋₄ alkyl, or
k) C₁₋₄ alkyl-S—C₁₋₄ alkyl;
R₃₂₀ is independently selected from
a) H, or
b) substituted alkyl;
Each R₃₂₅, R₃₂₆, R₃₂₇, and R₃₂₈ is independently selected from
a) H,
b) C₁-C₆alkyl,
c) substituted alkyl,
d) halo, or
e) R₃₂₅ and R₃₂₆ or R₃₂₇ and R₃₂₈ together are ═O, ═S, or ═N—R₃₃₂, or
f) one of R₃₂₅ or R₃₂₆ and R₃₀₃, when Z₄ is —N(R₃₀₃)—, together with the carbon and nitrogen atoms to which they are bound form a 5-7 membered het containing one or more heteroatoms selected from O, S, or N, the het being optionally substituted with one or more of R₂, ═O, or ═S;
R₃₃₀ is
a) H, or
b) alkyl, or
c) substituted alkyl;
R₃₃₁ is
a) R₃₃₂,
b) Cl,
c) NH₂,
d) OH,
e) NHC₁-C₄alkyl, or
f) R₃₁₅;
R₃₃₂ is
a) H,
b) C₁-C₄alkyl,
c) OC₁-C₄alkyl,
d) SC₁-C₄alkyl, or
e) NHC₁-C₄alkyl;
R₃₃₃ is
a) F, or
b) R₃₃₂;
R₅₀₀ and R₅₀₃ are each and independently
(a) H,
(b) halo,
(c) C₁-C₈ alkyl,
(d) C₃-C₆ cycloalkyl,
(e) —(CH₂)ᵢ—OR₅₁₁, or
(f) —C(═O)—R₅₄₁;
R₅₀₁ and R₅₀₂ are each and independently
(a) hydrogen atom,
(b) C₁-C₈ alkyl,
(c) C₁-C₈ alkoxy,
(d) C₁-C₈ alkylthio,
(e) —(CH₂)ᵢ—OR₅₅₁,
(f) —O—(CH₂)ᵢ—OR₅₅₁,
(g) —NR₅₄₂R₅₅₂,
(h) —C(═O)—NR₅₄₂R₅₅₂,
(i) —(CH₂)ᵢ—C(═O)—R₅₄₁,
or R₅₀₁, and R₅₀₂ together form
(j) ═O,
(k) ═NR₅₄₃,
(l) ═S,
(m) ═CR₅₄₄R₅₅₄, or
(n) an unsaturated or saturated 5- or 6-membered hetero ring having 1-3 hetero atoms selected from the group consisting of a nitrogen atom, an oxygen atom and a sulfur atom;
R₅₁₁ and R₅₁₂ are each and independently
(a) hydrogen atom,
(b) C₁-C₈ alkyl;
R₅₄₁ is
(a) hydrogen atom,
(b) —(CH₂)ₘ—OH,
(c) C₁-C₈ alkyl,
(d) C₁-C₈ alkoxy, or
(e) —O—CH₂—O—C(═O)—R₅₁₁;
R₅₄₂ and R₅₅₂ are each and independently
(a) hydrogen atom,
(b) —(CH₂)ᵢ—OH,
(c) C₁-C₈ alkyl,
(d) —C(═O)—R₅₄₁,
(e) —C(═O)'NR₅₁₁R₅₁₂,
(f) —(CH₂)_{q}-phenyl, or
or R₅₄₂ and R₅₅₂ together form a pyrrolidino group, a piperidino group, a piperazino group, a morpholino group, or a thiomorpholino group, each of which may be substituted by C₁-C₈ alkyl or —(CH₂)ᵢ—OH;
R₅₄₃ is
(a) H,
(b) —OR₅₅₁,
(c) C₁-C₈ alkyl,
(d) C₁-C₈ alkoxy,
(e) —(CH₂)_{q}-phenyl,
(f) —NR₅₄₂R₅₅₂,
(g) —NH—C(═NH)—NH₂, or
(h) [1,2,4]triazol-4-yl;
R₅₄₄ and R₅₅₄ are each and independently
(a) H,
(b) C₁-C₈ alkyl,
(c) —C(═O)—R₅₄₁, or
(d) —(CH₂)_{q}-phenyl;
R₅₅₁ is
(a) H,
(b) C₁-C₈ alkyl,
(c) C₁-C₈ alkyl substituted with 1-3 halo,
(d) —(CH₂)ᵢ—OR₁₁₁,
(e) —(CH₂)ᵢ—C(═O)—R₅₄₁, or
(f) —C(═O)—(CH₂)ᵢ—OR₅₄₄;
R₆₀₀ is
a) H,
b) C₁-C₄alkyl
c) het,
d) (CH₂)_{b}C(O)OC₁-C₄alkyl,
e) (CH₂)_{b}C(O)C₁-C₄alkyl, or
f) aryl;
R₆₀₁ and R₆₀₂ are each independently
a) H,
b) C₁-C₄alkyl,
c) het,
d) C₃-C₆cycloalkyl,
e) aryl,
f) OC₁-C₄alkyl,
g) C(O)OC₁-C₄alkyl; or
h) R₆₀₁ and R₆₀₂ taken together along with the carbon atom to which they attach form a C₃-C₆cycloalkyl;
Each R₇₀₀, R₇₀₁, R₇₀₂, R₇₀₃, R₇₀₄, and R₇₀₅ is independently selected from
a) H,
b) C₁₋₄ alkyl optionally substituted with 1-3 halo, ═O, ═S, —OH
c) C(O)NH₂,
d) —CN,
e) aryl,
f) substituted aryl,
g) het,
h) substituted het,
i) C(O)OH,
j) C(O)OC₁₋₄ alkyl, or
k) R₇₀₀ and R₇₀₁ form ═O or ═S, or
l) R₇₀₂ and R₇₀₃ form ═O or ═S, or
m) R₇₀₄ and R₇₀₅ form ═O or ═S;
a is 1 or 2;
b is 0 or 1;
h is 1, 2, or 3;
i is 0, 1, or 2;
j is 0 or 1;
k is 3, 4, or 5;
l is 2 or 3;
m is 2, 3, 4 or 5;
n is 0, 1, 2, 3, 4, or 5;
p is 0, 1, 2, 3, 4, or 5; with the proviso that n and p together are 1, 2, 3, 4, or 5;
q is 1, 2, 3, or 4;
t is 0, 1, 2, 3, 4, 5, or 6; and
w is 0, 1, 2, or 3.

9. The compound of claim 8, wherein each R₂ is independently selected from H, or F.

10. The compound of claim 8, wherein B is

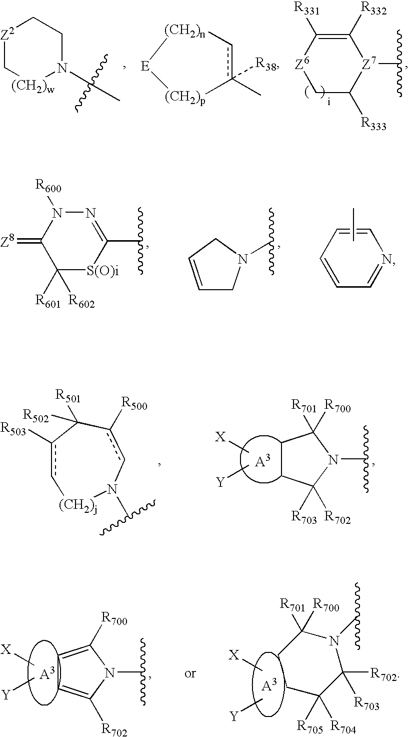

11. A compound of claim 2 which is
(5R)-3-(3-fluoro-4-morpholin-4-ylphenyl)-2-oxo-1,3-oxazolidine-5-carbonitrile,
(5R)-3-[3-fluoro-4-(5-methyl-1,3,4-thiadiazol-2-yl)phenyl]-2-oxo-1,3-oxazolidine-carbonitrile,
(5R)-3-[4-(1,1-dioxidothiomorpholin-4-yl)-3-fluorophenyl]-2-oxo-1,3-oxazolidine-5-carbonitrile,
(5R)-3-[4-(1,1-dioxidothiomorpholin-4-yl)-3,5-difluorophenyl]-2-oxo-1,3-oxazolidine-5-carbonitrile, or
(5R)-3-(3-fluoro-4-pyridin-4-ylphenyl)-2-oxo-1,3-oxazolidine-5-carbonitrile.
